SEQUENCE LISTING (26 sequences)

SEQ ID NO: 1 - DNA, 20 bp, Rattus rattus
ccccagtttgtggtctgcca

SEQ ID NO: 2 - DNA, 18 bp, Rattus rattus
gctaaaggtgaagatcta

SEQ ID NO: 3 - DNA, 28 bp, Rattus rattus
ttccttgggcagtagacctgactgtgag

SEQ ID NO: 4 - DNA, 28 bp, Rattus rattus
gtgaccgtggagtggcacccccacacac

SEQ ID NO: 5 - DNA, 1000 bp, Rattus rattus
gtccccaaatattatcaggaaaaggattgatcatttatggctgtacgatcggcacctaacagagcttagtaaaatagatttaatagctatctttggaaagaattaaatgaatgaaaatttgtgtgcattgatctcagttatttccagaatactgcaatatgatatgtatacttttcctttttgttttttgtttgtttgacacttttcttaacttgacttttccctccctccattttccccaggtagcttcgccaaaatggctgtccccttgccatctaccctgagactcagttctgcacctgatgaaattcagcacccgcacatcaaattttccgagtggaaatttaagctgtttagggtgagatcctttgaaaaggcacccgaagaagcacaaaaagagaaggattcctcagaagggaaaccttgtctcgagcagtctccagtagttctagataaccctgggggtcagaattcagttctgactcaacgagcattgaaactccatcctaaattttcaaagaaattccatgttgatgggaagtcaagcgacaaagcaattcaccaagccaggcttagacacttctgccgcatctgtggcaatcacttcaagagtgacgggcacaaccggagatacccagtccacgggcccgtggacgctaaaactcaaagccttttccgaaagaaggaaaaaagagtcacgtcctggccagatctcattgccagagttttccggattgatgtgaagtcagatgttgactccatccaccccactgaattctgccataactgttggagcattatgcacaggaagttcggcagtgctcacagtcaggtctactgcccaaggaatgtgaccgtggagtggcacccccacacaccgtcctgtgacatctgctttactgcccatcggggactgaagaggaagagacatcagcccaacgtgcagctcagcaagaaactaaaaactgtgctcaaccatgctagacgggaccgtcgcaag

SEQ ID NO: 6 - DNA, 200 bp, Rattus rattus
tgcacaggaagttcggcagtgctcacagtcaggtctactgcccaaggaatgtgaccgtggagtggcacccccacacaccgtcctgtgacatctgctttactgcccatcggggactgaagaggaagagacatcagcccaacgtgcagctcagcaagaaactaaaaactgtgctcaaccatgctagacgggaccgtcgcaag

SEQ ID NO: 7 - DNA, 28 bp, Rattus rattus
acgtggtatatagccgaggaaaaagtgt

SEQ ID NO: 8 - DNA, 28 bp, Rattus rattus
ataccacgtcaatggaatggccatatct

SEQ ID NO: 9 - DNA, 28 bp, Rattus rattus
ttaagggccatgaagatgaggatgctac

SEQ ID NO: 10 - DNA, 28 bp, Rattus rattus
cagcaagaccggaagccttccagtcagt

SEQ ID NO: 11 - DNA, 28 bp, Rattus rattus
ttgtcgattttggaaggattgagggccc

SEQ ID NO: 12 - DNA, 28 bp, Rattus rattus
atgcaggaagagctgcagaagtggaaga

SEQ ID NO: 13 - DNA, 28 bp, Rattus rattus
tacacaagtccttctccaggagctagaa

SEQ ID NO: 14 - DNA, 28 bp, Rattus rattus
acaaagcttatgaaggtcttagtgaaaa

SEQ ID NO: 15 - protein, 35 aa, Artificial Sequence
  COMPLETELY ARTIFICIAL
DRSNLSRQSGSLTRERGTLARRSDHLTTHKTSLKD

SEQ ID NO: 16 - protein, 35 aa, Artificial Sequence
  COMPLETELY ARTIFICIAL
QNATRIKRSDALSRQSGHLSRRSADLTEDRANLSR

SEQ ID NO: 17 - protein, 35 aa, Artificial Sequence
  COMPLETELY ARTIFICIAL
RSDNLSRDSSTRKKNSGNLDKQSGALARRSDALAR

SEQ ID NO: 18 - protein, 35 aa, Artificial Sequence
  COMPLETELY ARTIFICIAL
QSGNLARRSDSLSVQSADRTKRSDTLSTDRKTRIN

SEQ ID NO: 19 - protein, 35 aa, Artificial Sequence
  COMPLETELY ARTIFICIAL
TSGNLTRQSGNLARLKQNLDADRSHLTRRLDNRTA

SEQ ID NO: 20 - protein, 35 aa, Artificial Sequence
  COMPLETELY ARTIFICIAL
DRSDLSRQSGNLARRSDTLSEQRQHRTTQNATRIK

SEQ ID NO: 21 - protein, 35 aa, Artificial Sequence
  COMPLETELY ARTIFICIAL
RSDHLSAQSGHLSRDSESLNATSSNLSRDRSSRKR

SEQ ID NO: 22 - protein, 35 aa, Artificial Sequence
  COMPLETELY ARTIFICIAL
QSGSLTRQSSDLRRQRTHLTQQSGHLQRQSGDLTR

SEQ ID NO: 23 - protein, 35 aa, Artificial Sequence
  COMPLETELY ARTIFICIAL
QSGDLTRSSSDRKKDSSDRKKRSDNLSTDNSNRIN

SEQ ID NO: 24 - protein, 35 aa, Artificial Sequence
  COMPLETELY ARTIFICIAL
TSGHLSRQSGNLARHLGNLKTQSSDLSRQSGNRTT

SEQ ID NO: 25 - DNA, 300 bp, Rattus rattus
tatcatccacggagggaaaacgccaaacaatgagctttccgataagatttatatcatgtctgtcgcttgcaagaataacaaaaaagttactttccgttgtacagagaaagacttagtaggagatgtccctgaagccagatatggccattccattgacgtggtatatagccgaggaaaaagtgttggtgttctctttggaggacggtcatacatgccttctacccaaagaaccacagaaaaatggaatagtgtagctgattgcctaccccatgttttcttggtagattttgaatttgggtg

SEQ ID NO: 26 - DNA, 300 bp, Rattus rattus; identical to SEQ ID NO: 25

(19) United States
(12) Patent Application Publication
(10) Pub. No.: US 2011/0016543 A1
(43) Pub. Date: Jan. 20, 2011

(54) GENOMIC EDITING OF GENES INVOLVED IN INFLAMMATION

(75) Inventors: Edward Weinstein, St. Louis, MO (US); Xiaoxia Cui, St. Louis, MO (US); Phil Simmons, St. Louis, MO (US)

Correspondence Address: POLSINELLI SHUGHART PC, 700 W. 47TH STREET, SUITE 1000, KANSAS CITY, MO, 64112-1802, US

(73) Assignee: SIGMA-ALDRICH CO., St. Louis, MO (US)

(21) Appl. No.: 12/842,999

(22) Filed: Jul. 23, 2010

Related U.S. Application Data
(63) Continuation-in-part of application No. 12/592,852, filed on Dec. 3, 2009.
(60) Provisional application No. 61/343,287, filed on Apr. 26, 2010.
(60) Provisional application No. 61/323,702, filed on Apr. 13, 2010.
(60) Provisional application No. 61/323,719, filed on Apr. 13, 2010.
(60) Provisional application No. 61/323,698, filed on Apr. 13, 2010.
(60) Provisional application No. 61/309,729, filed on Mar. 2, 2010.
(60) Provisional application No. 61/308,089, filed on Feb. 25, 2010.
(60) Provisional application No. 61/336,000, filed on Jan. 14, 2010.
(60) Provisional application No. 61/263,904, filed on Nov. 24, 2009.
(60) Provisional application No. 61/263,696, filed on Nov. 23, 2009.
(60) Provisional application No. 61/245,877, filed on Sep. 25, 2009.
(60) Provisional application No. 61/232,620, filed on Aug. 10, 2009.
(60) Provisional application No. 61/228,419, filed on Jul. 24, 2009.
(60) Provisional application No. 61/200,985, filed on Dec. 4, 2008.
(60) Provisional application No. 61/205,970, filed on Jan. 26, 2009.

(51) Int. Cl. G01N 33/00; A01K 67/00; C12N 5/10

(52) U.S. Cl. 800/3; 800/13; 800/15; 800/17; 800/16; 800/14; 435/325; 435/351; 435/350; 435/352; 435/363; 435/353

(57) ABSTRACT

The present invention provides genetically modified animals and cells comprising edited chromosomal sequences encoding inflammation-related proteins. In particular, the animals or cells are generated using a zinc finger nuclease-mediated editing process. Also provided are methods of assessing the effects of agents in genetically modified animals and cells comprising edited chromosomal sequences encoding inflammation-related proteins.

### CROSS-REFERENCE TO RELATED APPLICATIONS

This application claims the priority of U.S. provisional application No. 61/343,287, filed Apr. 26, 2010, U.S. provisional application No. 61/323,702, filed Apr. 13, 2010, U.S. provisional application No. 61/323,719, filed Apr. 13, 2010, U.S. provisional application No. 61/323,698, filed Apr. 13, 2010, U.S. provisional application No. 61/309,729, filed Mar. 2, 2010, U.S. provisional application No. 61/308,089, filed Feb. 25, 2010, U.S. provisional application No. 61/336,000, filed Jan. 14, 2010, U.S. provisional application No. 61/263,904, filed Nov. 24, 2009, U.S. provisional application No. 61/263,696, filed Nov. 23, 2009, U.S. provisional application No. 61/245,877, filed Sep. 25, 2009, U.S. provisional application No. 61/232,620, filed Aug. 10, 2009, U.S. provisional application No. 61/228,419, filed Jul. 24, 2009, and is a continuation in part of U.S. non-provisional application Ser. No. 12/592,852, filed Dec. 3, 2009, which claims priority to U.S. provisional 61/200,985, filed Dec. 4, 2008 and U.S. provisional application 61/205,970, filed Jan. 26, 2009, all of which are hereby incorporated by reference in their entirety.

### FIELD OF THE INVENTION

The invention generally relates to genetically modified animals or cells comprising at least one edited chromosomal sequence encoding inflammation-related proteins. In particular, the invention relates to the use of a zinc finger nuclease-mediated process to edit chromosomal sequences encoding inflammation-related proteins.

### BACKGROUND OF THE INVENTION

Inflammation is part of the complex biological response of vascular tissues to harmful stimuli, such as pathogens, damaged cells, or irritants. Inflammation is a protective attempt by the organism to remove the injurious stimuli and to initiate the healing process. A large variety of proteins are involved in inflammation, and any one of them is open to a genetic mutation which impairs or otherwise dysregulates the normal function and expression of that protein. Without inflammation, wounds and infections would never heal. However, chronic inflammation can also lead to a host of diseases. Examples of disorders associated with inflammation include: acne vulgaris, asthma, hay fever, atheroscloris, autoimmune diseases, chronic inflammation, chronic prostatitis, glomerulonephritis, hypersensitivities, inflammatory bowel diseases, pelvic inflammatory disease, reperfusion injury, rheumatoid arthritis, transplant rejection, vasculitis, interstitial cystitis. It is for that reason that inflammation is normally closely regulated by the body. What are needed are animal models with these proteins genetically modified to provide research tools that allow the elucidation of mechanisms underlying development and progression of inflammation.

### SUMMARY OF THE INVENTION

One aspect of the present disclosure encompasses a genetically modified animal comprising at least one edited chromosomal sequence encoding an inflammation-related protein.

A further aspect provides a non-human embryo comprising at least one RNA molecule encoding a zinc finger nuclease that recognizes a chromosomal sequence encoding an inflammation-related protein, and, optionally, at least one donor polynucleotide comprising a sequence encoding an inflammation related protein.

Yet an additional aspect encompasses a method for assessing the effect of mutant inflammation-related proteins on the progression or symptoms of a disease state associated with inflammation-related proteins in an animal. The method comprises comparing a wild type animal to a genetically modified animal comprising at least one edited chromosomal sequence encoding an inflammation-related protein, and measuring a phenotype associated with the disease state.

Another aspect encompasses a method for assessing the effect of an agent on progression or symptoms of inflammation. The method comprises (a) contacting a genetically modified animal comprising at least one edited chromosomal sequence encoding an inflammation-related protein with the agent, measuring an inflammation-related phenotype, and (c) comparing results of the inflammation-related phenotype in (b) to results obtained from a control genetically modified animal comprising said edited chromosomal sequence encoding an inflammation-related protein not contacted with the agent.

Other aspects and features of the disclosure are described more thoroughly below.

### REFERENCE TO COLOR FIGURES

The application file contains at least one figure executed in color. Copies of this patent application publication with color figures will be provided by the Office upon request and payment of the necessary fee.

### BRIEF DESCRIPTION OF THE FIGURES

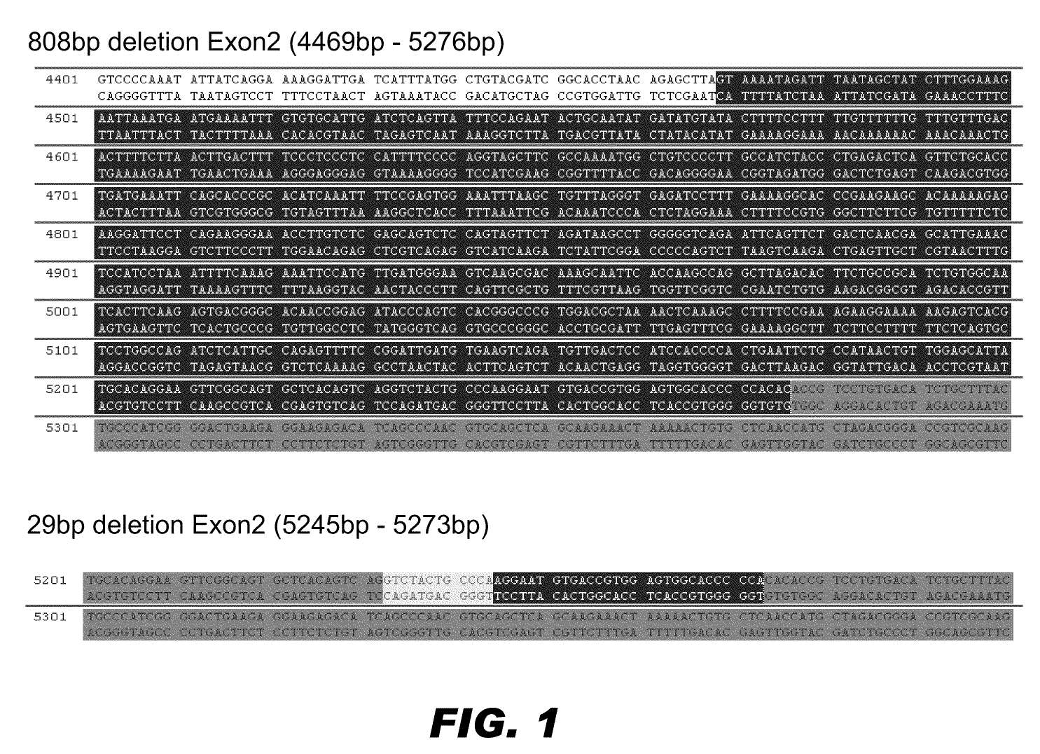

FIG. 1 presents the DNA sequences of edited Rag1 loci in two animals. The upper sequence (SEQ ID NO:5) has a 808 bp deletion in exon 2, and the lower sequence (SEQ ID NO:6) has a 29 bp deletion in exon 2. The exon sequence is shown in green; the target site is presented in yellow, and the deletions are shown in dark blue.

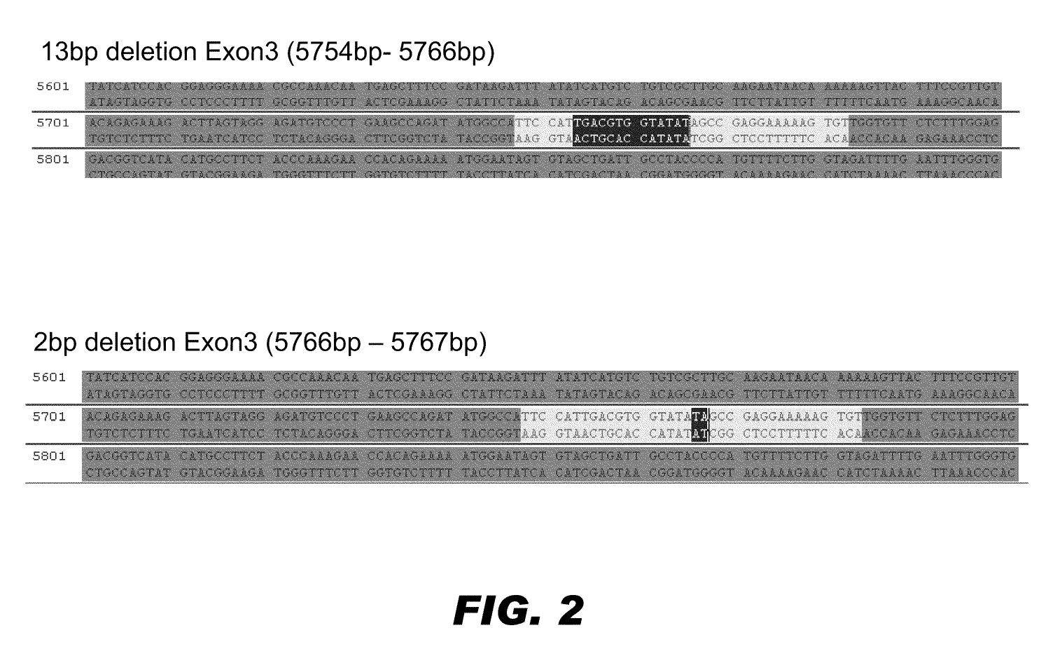

FIG. 2 presents the DNA sequences of edited Rag2 loci in two animals. The upper sequence (SEQ ID NO: 25) has a 13 bp deletion in the target sequence in exon 3, and the lower sequence (SEQ ID NO:26) has a 2 bp deletion in the target sequence in exon 2. The exon sequence is shown in green; the target site is presented in yellow, and the deletions are shown in dark blue.

### DETAILED DESCRIPTION OF THE INVENTION

The present disclosure provides a genetically modified animal or animal cell comprising at least one edited chromosomal sequence encoding a protein associated with inflammation. The edited chromosomal sequence may be (1) inactivated, (2) modified, or (3) comprise an integrated sequence. An inactivated chromosomal sequence is altered such that a functional protein is not made. Thus, a genetically modified animal comprising an inactivated chromosomal sequence may be termed a “knock out” or a “conditional knock out.” Similarly, a genetically modified animal comprising an integrated sequence may be termed a “knock in” or a “conditional knock in.” As detailed below, a knock in animal may be a humanized animal. Furthermore, a genetically modified animal comprising a modified chromosomal sequence may comprise a targeted point mutation(s) or other modification such that an altered protein product is produced. The chromosomal sequence encoding the protein associated with inflammation generally is edited using a zinc finger nuclease-mediated process. Briefly, the process comprises introducing into an embryo or cell at least one RNA molecule encoding a targeted zinc finger nuclease and, optionally, at least one accessory polynucleotide. The method further comprises incubating the embryo or cell to allow expression of the zinc finger nuclease, wherein a double-stranded break introduced into the targeted chromosomal sequence by the zinc finger nuclease is repaired by an error-prone non-homologous end-joining DNA repair process or a homology-directed DNA repair process. The method of editing chromosomal sequences encoding a protein associated with inflammation using targeted zinc finger nuclease technology is rapid, precise, and highly efficient.

### (I) Genetically Modified Animals.

One aspect of the present disclosure provides a genetically modified animal in which at least one chromosomal sequence encoding an inflammation-related protein has been edited. For example, the edited chromosomal sequence may be inactivated such that the sequence is not transcribed and/or a functional inflammation-related protein is not produced. Alternatively, the chromosomal sequence may be edited such that the sequence is over-expressed and a functional inflammation-related protein is over-produced. The edited chromosomal sequence may also be modified such that it codes for an altered inflammation-related protein. For example, the chromosomal sequence may be modified such that at least one nucleotide is changed and the expressed inflammation-related protein comprises at least one changed amino acid residue (missense mutation). The chromosomal sequence may be modified to comprise more than one missense mutation such that more than one amino acid is changed. Additionally, the chromosomal sequence may be modified to have a three nucleotide deletion or insertion such that the expressed inflammation-related protein comprises a single amino acid deletion or insertion, provided such a protein is functional. The modified inflammation-related protein may have altered substrate specificity, altered enzyme activity, altered kinetic rates, and so forth. Furthermore, the edited chromosomal sequence encoding an inflammation-related protein may comprise a sequence encoding an inflammation-related protein integrated into the genome of the animal. The chromosomally integrated sequence may encode an endogenous inflammation-related protein normally found in the animal, or the integrated sequence may encode an orthologous inflammation-related protein, or combinations of both. The genetically modified animal disclosed herein may be heterozygous for the edited chromosomal sequence encoding an inflammation-related protein. Alternatively, the genetically modified animal may be homozygous for the edited chromosomal sequence encoding an inflammation-related protein.

In one embodiment, the genetically modified animal may comprise at least one inactivated chromosomal sequence encoding an inflammation-related protein. The inactivated chromosomal sequence may include a deletion mutation (i.e., deletion of one or more nucleotides), an insertion mutation (i.e., insertion of one or more nucleotides), or a nonsense mutation (i.e., substitution of a single nucleotide for another nucleotide such that a stop codon is introduced). As a consequence of the mutation, the targeted chromosomal sequence is inactivated and a functional inflammation-related protein is not produced. The inactivated chromosomal sequence comprises no exogenously introduced sequence. Such an animal may be termed a “knockout.” Also included herein are genetically modified animals in which two, three, or more chromosomal sequences encoding inflammation-related proteins are inactivated.

In another embodiment, the genetically modified animal may comprise at least one edited chromosomal sequence encoding an inflammation-related protein such that the sequence is over-expressed and a functional inflammation-related protein is over-produced. For example, the regulatory regions controlling the expression of the inflammation-related protein may be altered such that the inflammation-related protein is over-expressed.

In yet another embodiment, the genetically modified animal may comprise at least one chromosomally integrated sequence encoding an inflammation-related protein. For example, an exogenous sequence encoding an orthologous or an endogenous inflammation-related protein may be integrated into a chromosomal sequence encoding an inflammation-related protein such that the chromosomal sequence is inactivated, but wherein the exogenous sequence encoding the orthologous or endogenous inflammation-related protein may be expressed or over-expressed. In such a case, the sequence encoding the orthologous or endogenous inflammation-related protein may be operably linked to a promoter control sequence. Alternatively, an exogenous sequence encoding an orthologous or endogenous inflammation-related protein may be integrated into a chromosomal sequence without affecting expression of a chromosomal sequence. For example, an exogenous sequence encoding an inflammation-related protein may be integrated into a “safe harbor” locus, such as the Rosa26 locus, HPRT locus, or AAV locus, wherein the exogenous sequence encoding the orthologous or endogenous inflammation-related protein may be expressed or over-expressed. In one iteration of the disclosure, an animal comprising a chromosomally integrated sequence encoding an inflammation-related protein may be called a “knock-in”, and it should be understood that in such an iteration of the animal, no selectable marker is present. The present disclosure also encompasses genetically modified animals in which 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or more sequences encoding inflammation-related proteins are integrated into the genome.

The chromosomally integrated sequence encoding an inflammation-related protein may encode the wild type form of the inflammation-related protein. Alternatively, the chromosomally integrated sequence encoding an inflammation-related protein may comprise at least one modification such that an altered version of the inflammation-related protein is produced. In some embodiments, the chromosomally integrated sequence encoding an inflammation-related protein comprises at least one modification such that the altered version of the protein causes inflammation. In other embodiments, the chromosomally integrated sequence encoding an inflammation-related protein comprises at least one modification such that the altered version of the inflammation-related protein protects against inflammation.

In an additional embodiment, the genetically modified animal may be a “humanized” animal comprising at least one chromosomally integrated sequence encoding a functional human inflammation-related protein. The functional human inflammation-related protein may have no corresponding ortholog in the genetically modified animal. Alternatively, the wild-type animal from which the genetically modified animal is derived may comprise an ortholog corresponding to the functional human inflammation-related protein. In this case, the orthologous sequence in the “humanized” animal is inactivated such that no functional protein is made and the “humanized” animal comprises at least one chromosomally integrated sequence encoding the human inflammation-related protein. For example, a humanized animal may comprise an inactivated abat sequence and a chromosomally integrated human ABAT sequence. Those of skill in the art appreciate that “humanized” animals may be generated by crossing a knock out animal with a knock in animal comprising the chromosomally integrated sequence.

In yet another embodiment, the genetically modified animal may comprise at least one edited chromosomal sequence encoding an inflammation-related protein such that the expression pattern of the protein is altered. For example, regulatory regions controlling the expression of the protein, such as a promoter or transcription binding site, may be altered such that the inflammation-related protein is over-produced, or the tissue-specific or temporal expression of the protein is altered, or a combination thereof. Alternatively, the expression pattern of the inflammation-related protein may be altered using a conditional knockout system. A non-limiting example of a conditional knockout system includes a Cre-lox recombination system. A Cre-lox recombination system comprises a Cre recombinase enzyme, a site-specific DNA recombinase that can catalyze the recombination of a nucleic acid sequence between specific sites (lox sites) in a nucleic acid molecule. Methods of using this system to produce temporal and tissue specific expression are known in the art. In general, a genetically modified animal is generated with lox sites flanking a chromosomal sequence, such as a chromosomal sequence encoding an inflammation-related protein. The genetically modified animal comprising the lox-flanked chromosomal sequence encoding an inflammation-related protein may then be crossed with another genetically modified animal expressing Cre recombinase. Progeny animals comprising the lox-flanked chromosomal sequence and the Cre recombinase are then produced, and the lox-flanked chromosomal sequence encoding an inflammation-related protein is recombined, leading to deletion or inversion of the chromosomal sequence encoding the protein. Expression of Cre recombinase may be temporally and conditionally regulated to effect temporally and conditionally regulated recombination of the chromosomal sequence encoding an inflammation-related protein.

### (a) Inflammation-Related Proteins

The present disclosure comprises editing of any chromosomal sequences that encode proteins associated with inflammation. The inflammation-related proteins are typically selected based on an experimental association of the inflammation-related protein to an inflammation disorder. For example, the production rate or circulating concentration of an inflammation-related protein may be elevated or depressed in a population having an inflammation disorder relative to a population lacking the inflammation disorder. Differences in protein levels may be assessed using proteomic techniques including but not limited to Western blot, immunohistochemical staining, enzyme linked immunosorbent assay (ELISA), and mass spectrometry. Alternatively, the inflammation-related proteins may be identified by obtaining gene expression profiles of the genes encoding the proteins using genomic techniques including but not limited to DNA microarray analysis, serial analysis of gene expression (SAGE), and quantitative real-time polymerase chain reaction (Q-PCR).

By way of non-limiting example, inflammation-related proteins include but are not limited to the proteins listed in Table A.

TABLE A

| Edited Chromosomal | |
|---|---|
| Sequence | Encoded Protein |
| A4GALT | CD77 |
| ABL1 | ABL1 |
| ACE | angiotensin converting |
| | enzyme, CD143 |
| ACIN1 | Acinus |
| ADAM17 | CD156b, TNFa converting |
| | enzyme |
| ADAM8 | CD156a, ADAM8 |
| ADCY1 | adenylyl cyclase I |
| ADCY2 | adenylyl cyclase II |
| ADCY4 | adenylyl cyclase IV |
| ADCY5 | adenylyl cyclase V |
| ADCY6 | adenylyl cyclase VI |
| ADORA1 | adenosine receptor A1 |
| ADORA2A | adenosine receptor A2A |
| ADORA2B | adenosine receptor A2B |
| ADORA3 | adenosine receptor A3 |
| ADRA2A | alpha2-adrenergic receptor A |
| ADRA2C | alpha2-adrenergic receptor C |
| ADRB2 | beta2-adrenergic receptor |
| ADRBK1 | GRK2, G-protein receptor |
| | kinase 2 |
| AGER | RAGE |
| AKAP5 | AKAP5 |
| AKR1C3 | PGFS, F-prostanoid |
| | synthase |
| AKT1 | AKT |
| AKT2 | AKT |
| AKT3 | AKT |
| ALCAM | CD166, activated leukocyte |
| | cell adhesion molecule |
| ALOX12 | ALOX12 |
| ALOX12B | ALOX13 |
| ALOX15 | ALOX15 |
| ALOX15B | ALOX16 |
| ALOX5 | ALOX5 |
| ALOX5AP | ALOX5AP |
| ALS2 | ALS2 |
| ANPEP | CD13 |
| APAF1 | Apaf1 |
| ARAF | A-Raf |
| ARHGAP1 | Cdc42 |
| ATF1 | ATF-1 |
| ATF2 | ATF-2 |
| ATF3 | ATF3 |
| ATF4 | ATF4 |
| B3GAT1 | CD57 |
| BAD | BAD |
| BAK1 | BAK |
| BAX | BAX |
| BBC3 | BBC3 |
| BCAR1 | CAS |
| BCL10 | BCL10 |
| BCL2 | Bcl-2 |
| BCL2A1 | Bfl-1 |
| BCL2L1 | Bcl-XL |
| BCL2L10 | BCL2L10 |
| BCL2L11 | BCL2L11 |
| BCL2L12 | BCL2L12 |
| BCL2L13 | BCL2L13 |
| BCL2L14 | Bcl-GS |
| BCL2L2 | Bcl-2 like 2 |
| BCL3 | Bcl3 |
| BCL6 | Bcl6 |
| BID | Bid |
| BIK | BCL2-interacting killer |
| BIRC2 | cIAP |
| BIRC3 | cIAP |
| BLNK | BLNK, B cell linker protein |
| BLR1 | B-lymphocyte |
| | chemoattractant receptor, |
| | CXCR5, CD185 |
| BMP2 | bone morphogenetic |
| | protein 2 |
| BMP4 | bone morphogenetic |
| | protein 4 |
| BMPR1A | BMP receptor 1A |
| BMPR1B | BMP receptor 1B |
| BMPR2 | BMP receptor 2 |
| BPI | BPI |
| BRAF | B-Raf |
| BTK | Bruton tyrosine kinase |
| BTRC | beta-TrCP |
| C190RF10 | interleukin 25, IL-27w |
| C1QA | C1q |
| C1QB | C1q |
| C1QC | C1q |
| C1R | C1r |
| C1S | C1s |
| C2 | C2 |
| C3 | C3 |
| C3AR1 | C3AR |
| C48 | C4B (basic) |
| C4BPA | C4BPalpha |
| C4BPB | C4BPbeta |
| C5 | C5 |
| C5AR1 | C5AR |
| C6 | C6 |
| C7 | C7 |
| C8A | C8A |
| C8B | C8B |
| C8G | C8G |
| C9 | C9 |
| C90RF26 | interleukin 33 |
| CABIN1 | CABIN1 |
| CAMK1D | CaMK I |
| CAMK4 | CaMK IV |
| CAPN1 | Calpain 1, large subunit |
| CAPN10 | Calpain 10, large subunit |
| CAPN2 | Calpain 2, large subunit |
| CAPNS1 | Calpain small subunit 1 |
| CARD10 | BIMP1 |
| CARD11 | CARMA1/BIMP3 |
| CARD12 | CARD12 |
| CARD14 | CARMA2/BIMP2 |
| CARD4 | NOD1 |
| CARD6 | CARD6 (predicted) |
| CARD8 | CARDINAL/CARD8 |
| CARD9 | CARMA3/CARD9 |
| CASP1 | Caspase 1 |
| CASP10 | Caspase 10 |
| CASP12 | Caspase 12 |
| CASP14 | Caspase 14 |
| CASP2 | Caspase 2 |
| CASP3 | Caspase 3 |
| CASP5 | Caspase 5 |
| CASP6 | Caspase 6 |
| CASP7 | Caspase 7 |
| CASP8 | Caspase 8 |
| CASP8AP2 | CASP8 associated protein 2 |
| CASP9 | Caspase 9 |
| CAT | catalase |
| CBL | CBL |
| CCBP2 | CCBP2 |
| CCL1 | CCL1 |
| CCL11 | CCL11 |
| CCL13 | CCL13 |
| CCL15 | CCL15 |
| CCL16 | CCL16 |
| CCL17 | CCL17 |
| CCL18 | CCL18 |
| CCL19 | CCL19 |
| CCL2 | CCL2 |
| CCL20 | CCL20 |
| CCL21 | CCL21 |
| CCL22 | CCL22 |
| CCL23 | CCL23 |
| CCL24 | CCL24 |
| CCL26 | CCL26 |
| CCL27 | CCL27 |
| CCL28 | CCL28 |
| CCL4 | CCL4 |
| CCL5 | CCL5 |
| CCL7 | CCL7 |
| CCL8 | CCL8 |
| CCND1 | cyclin D1 |
| CCR1 | CCR1 |
| Ccr2 | monocyte chemoattractant |
| | protein-1 (MCP1) |
| CCR3 | CCR3 |
| CCR4 | CCR4 |
| Ccr5 | C-C chemokine receptor |
| | type 5 (CCR5) |
| CCR6 | CCR6 |
| CCR7 | CCR7 |
| CCR8 | CCR8 |
| CCRL2 | CCRL2 |
| CD14 | CD14 |
| CD160 | CD160 |
| CD180 | Ly64, CD180 |
| CD1A | CD1A |
| CD1B | CD1B |
| CD1C | CD1C |
| CD1D | CD1D |
| CD1E | CD1E |
| CD2 | CD2 |
| CD207 | CD207 |
| CD209 | CD209, DC-SIGN |
| CD22 | CD22 |
| CD226 | CD226 |
| CD244 | 2B4 |
| CD28 | CD28 |
| CD300A | IRp60 |
| CD33 | CD33, Siglec-3 |
| CD34 | CD34 |
| CD36 | CD36, thrombospondin |
| | receptor |
| CD37 | CD37 |
| CD38 | CD38 |
| CD3E | CD3epsilon |
| CD3Z | CD3zeta |
| CD4 | CD4 |
| CD40 | CD40 |
| CD40LG | CD40L |
| CD44 | CD44 |
| CD46 | MCP |
| CD47 | CD47 |
| CD48 | CD48 |
| CD5 | CD5 |
| CD52 | CD52, B7-Ag |
| CD53 | CD53 |
| CD55 | DAF |
| CD58 | CD58 |
| CD59 | CD59 |
| CD68 | CD68, scavenger receptor |
| | D1 |
| CD74 | CD74 |
| CD79A | CD79A |
| CD79B | CD79B |
| CD80 | B7-1,CD80 |
| CD86 | B7-2,CD86 |
| CD8A | CD8A |
| CD8B1 | CD8B1 |
| CD9 | CD9 |
| CD96 | CD96 |
| CD97 | CD97 |
| CD99 | CD99 |
| CDC2 | Cdc2 |
| CDC37 | Cdc37 |
| CDH5 | cadherin 5 |
| CDKN1A | p21Cip1 |
| CDKN1B | p27Kip1 |
| CEACAM1 | CD55a |
| CEACAM3 | CD66d |
| CEACAM5 | CD66e |
| CEACAM6 | CD66c |
| CEACAM8 | CD66b |
| CEBPB | NF-IL6 |
| CFB | BF |
| CFD | DF |
| CFH | HF1 |
| CFI | IF |
| CFLAR | FLIP |
| CHUK | IKKalpha |
| CIAS1 | CIAS1 |
| CIITA | CIITA |
| CISH | CIS |
| CITED2 | Cbp/p300-interacting |
| | transactivator |
| CKLF | CKLF |
| CMA1 | CMA1 |
| COP1 | COP1 |
| COX1 | cytochrome c oxidase 1 or |
| | cyclooxygenase 1 (COX1) |
| COX2 | cytochrome c oxidase 2 |
| | (COX2) |
| CPAMD8 | CPAMD8 |
| CR1 | CR1 |
| CR2 | CR2 |
| CRADD | RAIDD |
| CREB1 | CREB |
| CREBBP | CBP/p300 |
| CREM | CREM |
| CRK | CRK |
| CRP | CRP |
| CRSP2 | DRIP150 |
| CSF1 | M-CSF |
| CSF1R | M-CSF Receptor |
| CSF2 | GM-CSF |
| CSF2RB | GM-CSF receptor, beta, |
| | low-affinity |
| CSF3 | G-CSF |
| CSF3R | G-CSF Receptor |
| CSK | Csk |
| CSNK2A1 | CK2 |
| CSNK2A2 | CK2 |
| CSNK2B | CK2 |
| CST7 | cystatin F |
| CTLA4 | Cytotoxic T-Lymphocyte |
| | Antigen 4 (CTLA4, CD152) |
| CTNNB1 | beta-catenin |
| CTNND1 | catenin delta 1 |
| CTSS | cathespin S |
| CTTN | cortactin |
| CX3CL1 | Chemokine (C—X3—C motif) |
| | ligand 1 (CX3CL1) |
| CX3CR1 | Chemokine (C—X3—C motif) |
| | receptor 1 (CX3CR1) |
| CXCL1 | CXCL1 |
| CXCL10 | CXCL10 |
| CXCL11 | CXCL11 |
| CXCL12 | CXCL12 |
| CXCL13 | CXCL13 |
| CXCL14 | CXCL14 |
| CXCL16 | CXCL16 |
| CXCL2 | CXCL2 |
| CXCL3 | CXCL3 |
| CXCL5 | CXCL5 |
| CXCL9 | CXCL9 |
| CXCR6 | CXCR6 |
| CYBB | cytochrome b-245, beta |
| CYCS | Cytochrome C |
| CYSLTR1 | CYSLTR |
| CYSLTR2 | CYSLTR |
| DAP | DAP |
| DAP3 | DAP3 |
| DAPK1 | DAPK1 |
| DAPP1 | DAPP1 |
| DARC | Duffy blood group, |
| | chemokine receptor |
| DAXX | Daxx |
| DDIT3 | CHOP |
| DDX58 | RIG-I |
| DFFA | ICAD |
| DFFB | CAD |
| DIABLO | Diablo |
| DPEP1 | DPEP |
| DPEP2 | DPEP |
| DPEP3 | DPEP |
| DPP4 | CD26 |
| DUSP1 | MKP1 |
| DUSP10 | MKP5 |
| DUSP2 | PAC1 |
| DUSP4 | MKP2 |
| DUSP6 | MKP1/2/3/4 |
| DUSP9 | MKP1/2/3/4 |
| EBI3 | interleukin 27 |
| EDNRA | endothelin receptor type A |
| EDNRB | endothelin receptor type B |
| EEF2K | eEF2K |
| EGF | Epidermal growth factor |
| | receptor |
| EGFR | EGF receptor |
| EIF2AK2 | PKR |
| EIF4E | eIF4E |
| ELA2 | ELA2 |
| ELK1 | Elk-1 |
| ENDOG | Endo G |
| ENG | CD105 |
| EP300 | CBP/p300 |
| ESR1 | ER |
| ETS1 | Ets |
| ETS2 | Ets |
| F11R | F11R |
| FADD | FADD |
| FAF1 | Fas associated factor 1 |
| FAIM | FAIM |
| FAS | Fas |
| FASLG | FasL |
| FCAR | IgA receptor |
| FCER1A | IgE receptor I, high affinity |
| FCER1G | FCER1g (Fc epsilon R1g) |
| FCER2 | IgE receptor II, CD23 |
| FCGR1C | CD64c, Fc gamma receptor |
| | 1c |
| FCGR2a | Low affinity |
| | immunoglobulin gamma Fc |
| | region receptor II-a |
| FCGR2A | FCGR2 |
| FCGR2B | IgG receptor IIB, (FCGR2B |
| | or CD32) |
| FCGR3A | FCGR3 |
| FGFR2 | Fibroblast growth factor |
| | receptor 2 (FGFR2) |
| FGA | Fibrinogen alpha chain |
| | (Fibrinogen I, FGA) |
| FKBP4 | FK506 binding protein 4, |
| | 59 kDa |
| FLT3 | Flt3 |
| FLT3LG | Flt3 ligand |
| FOS | c-Fos |
| FOSL1 | FOSL1 |
| FOXN1 | FOXN1 |
| FOXO1A | FKHR |
| FOXO3A | forkhead box O3A |
| FOXP3 | FOXP3 |
| FPRL1 | LXA4R |
| FPRL2 | LXA4R |
| FRAP1 | mTOR |
| FUT4 | CD15 |
| FYB | FYB |
| FYN | Fyn |
| GAB1 | GAB1 |
| GAS2 | Gas2 |
| GATA3 | GATA-3 |
| GGT1 | GGT1 |
| GLCCI1 | glucocorticoid induced |
| | transcript 1 |
| GMEB1 | glucocorticoid modulatory |
| | element binding protein 1 |
| GMEB2 | glucocorticoid modulatory |
| | element binding protein 2 |
| GPR44 | CRTH2 |
| GPX2 | glutathione peroxidase 2 |
| GPX3 | glutathione peroxidase 3 |
| GRAP2 | GADS, GRB2L |
| GRB2 | GRB2 |
| GRK4 | GRK4 |
| GZMA | Granzyme A |
| GZMB | Granzyme B |
| GZMH | Granzyme H |
| GZMM | Granzyme M |
| HDAC1 | HDAC1/2 |
| HDAC2 | HDAC1/2 |
| HINT1 | HINT1 |
| HLA-A | HLA-A |
| HLA-B | HLA-B |
| HLA-C | HLA-C |
| HLA-DMA | HLA-DMA |
| HLA-DMB | HLA-DMB |
| HLA-DOA | HLA-DOA |
| HLA-DPA1 | HLA-DPA1 |
| HLA-DPB1 | HLA-DPB1 |
| HLA-DQA1 | HLA-DQA1 |
| HLA-DQA2 | HLA-DQA2 |
| HLA-DQB2 | HLA-DQB2 |
| HLA-DRA | HLA-DRA |
| HLA-E | HLA-E |
| HLA-G | HLA-G |
| HMGB1 | HMGB1, AMPHOTERIN |
| HMGN1 | HMG-14 |
| HMMR | CD168, hyaluronan- |
| | mediated motility receptor |
| HRAS | Ras |
| HRH1 | HRH1 |
| HRH2 | HRH2 |
| HSP90AA1 | HSP90 |
| HSP90AB1 | HSP90 |
| HSP90B1 | Heat shock protein 90B |
| HSPB1 | HSP27 |
| HSPB2 | HSP27 |
| HSPD1 | heat shock 60 kDa protein 1 |
| | (chaperonin) |
| HTRA2 | HtrA2 |
| ICAM1 | ICAM1 |
| ICAM2 | ICAM2 |
| ICAM3 | ICAM3 |
| ICAM5 | ICAM5 |
| ICEBERG | ICEBERG |
| ICOS | ICOS |
| ICOSLG | ICOS-L |
| IFI16 | IFN-gamma inducible |
| | protein 16 |
| IFI30 | IFN-gamma inducible |
| | protein 30 |
| IFIH1 | MDA5 |
| IFNA1 | IFN-alpha |
| IFNA10 | IFNA10 |
| IFNA2 | IFNA2 |
| IFNA21 | IFNA21 |
| IFNA4 | IFNA4 |
| IFNA5 | IFN-alpha |
| IFNA6 | IFNA6 |
| IFNA8 | IFNA8 |
| IFNAR1 | IFNAR1 |
| IFNAR2 | IFNAR2 |
| IFNB1 | IFN-beta |
| IFNG | Interferon-gamma (IFN-) |
| IFNGR1 | IFN-gamma receptor alpha |
| IFNGR2 | IFN-gamma receptor beta |
| IFNK | IFN-kappa |
| IFNW1 | IFN-w |
| IGHA1 | Immunoglobulin heavy |
| | constant alpha 1 |
| IGLL1 | lambda5 |
| IGSF2 | CD101 |
| IGSF3 | CD101 |
| IKBKB | IKKbeta |
| IKBKG | NEMO/IKKG |
| IL-10 | Interleukin-10 (IL-10 or |
| | IL10), also known as |
| | human cytokine synthesis |
| | inhibitory factor (CSIF) |
| IL10RA | interleukin 10 receptor, |
| | alpha |
| IL10RB | interleukin 10 receptor, |
| | beta |
| IL11 | interleukin 11 |
| IL-12A | Subunit alpha of interleukin |
| | 12 |
| IL-12B | Subunit beta of interleukin |
| | 12 |
| IL12RB1 | IL12Rbeta1 |
| IL12RB2 | ILI2Rbeta2 |
| IL-13 | Interleukin 13 (IL-13) |
| IL13RA1 | IL13Ralpha1 |
| IL13RA2 | interleukin 13 receptor, |
| | alpha 2 |
| IL15 | interleukin 15 |
| IL15RA | IL15Ralpha |
| IL16 | interleukin 16 |
| IL17A | interleukin 17A |
| IL17B | interleukin 17B |
| IL-17C | Interleukin 17C |
| IL-17D | Interleukin 17D |
| IL-17F | Interleukin 17F |
| IL17RA | interleukin 17 receptor A |
| IL17RB | interleukin 17 receptor B |
| IL18 | interleukin 18 |
| IL18R1 | interleukin 18 receptor 1 |
| IL18RAP | IL18RAP |
| IL19 | interleukin 19 |
| IL1A | interleukin 1, alpha |
| IL-1B | Interleukin-1 beta (IL- |
| | 1 beta) |
| IL1F10 | IL1F10 |
| IL1F5 | interleukin 1 family, |
| | member 5 (delta) |
| IL1F6 | interleukin 1 family, |
| | member 6 (epsilon) |
| IL1F7 | IL1F7 |
| IL1F8 | IL1F8 |
| IL1F9 | interleukin 1 family, |
| | member 9 |
| IL1R1 | interleukin 1 receptor, type I |
| IL1R2 | IL-1R/TLR |
| IL1RAP | IL1-R-AP |
| IL1RL1 | IL1RL1 |
| IL1RL2 | IL1RL2 |
| IL1RN | IL-1RA |
| IL2 | interleukin 2 |
| IL20 | interleukin 20 |
| IL21 | interleukin 21 |
| IL21R | interleukin 21 receptor |
| IL22 | interleukin 22 |
| IL22RA1 | interleukin 22 receptor, |
| | alpha 1 |
| IL-23 | Interleukin 23 |
| IL23R | interleukin 23 receptor |
| IL24 | interleukin 24 |
| IL25 | interleukin 25 |
| IL26 | interleukin 26 |
| IL27RA | interleukin 27 receptor, |
| | alpha |
| IL28A | interleukin 28A (interferon, |
| | lambda 2) |
| IL28B | interleukin 28B (interferon, |
| | lambda 3) |
| IL29 | interleukin 29 (interferon, |
| | lambda 1) |
| IL2RA | interleukin 2 receptor, |
| | alpha |
| IL2RB | interleukin 2 receptor, beta |
| IL2RG | interleukin 2 receptor, |
| | gamma |
| IL3 | interleukin 3 |
| IL31 | interleukin 31 |
| IL31RA | IL31Ralpha |
| IL32 | interleukin 32 |
| IL3RA | IL3Ralpha |
| IL-4 | Interleukin-4 (IL-4) |
| IL4R | interleukin 4 receptor |
| IL5 | interleukin 5 |
| IL5RA | interleukin 5 receptor, |
| | alpha |
| IL6 | interleukin 6 |
| IL-6 | Interleukin-6 (IL-6) |
| IL6R | interleukin 6 receptor |
| IL6ST | GP130 |
| IL7 | interleukin 7 |
| IL7R | interleukin 7 receptor |
| IL8 | interleukin 8 |
| IL8RA | IL8Ralpha |
| IL8RB | IL8Rbeta |
| IL9 | interleukin 9 |
| ILF2 | IL-2 binding factor 2 |
| ILK | ILK |
| INPP5D | SHIP |
| INPPL1 | SHIP |
| INS | Insulin |
| IRAK1 | IRAK1 |
| IRAK2 | IRAK2 |
| IRAK3 | IRAK-M |
| IRAK4 | IRAK4 |
| IRF1 | IRF1 |
| IRF2 | IRF2 |
| IRF3 | IRF3 |
| IRF4 | IRF4 |
| IRF5 | IRF5 |
| IRF6 | IRF6 |
| IRF7 | IRF7 |
| IRF8 | IRF8 |
| ISGF3G | IRF9 |
| ITGA1 | Integrin A1, CD49a |
| ITGA2 | Integrin A2, CD49b |
| ITGA2B | integrin, alpha 2b |
| ITGA3 | Integrin A3, CD49c |
| ITGA4 | CD49D, VLA-4 |
| ITGA5 | Integrin 5, CD49e |
| ITGA6 | Integrin A6, CD49f |
| ITGAD | CD11c |
| ITGAE | Integrin alpha E, CD103 |
| ITGAL | CD11A |
| ITGAM | CD11B |
| ITGAV | Integrin AV, CD51 |
| ITGAX | CD11c |
| ITGB1 | CD29, fibronectin receptor |
| ITGB2 | CD18 |
| ITGB3 | Integrin B3, CD61 |
| ITGB4 | Integrin B4, CD104 |
| ITGB7 | Integrin B7, CD103b |
| ITK | IL2-inducible T-cell kinase |
| JAK1 | JAK1 |
| JAK2 | JAK2 |
| JAK3 | JAK3 |
| JAM2 | junctional adhesion |
| | molecule 2 |
| JAM3 | junctional adhesion |
| | molecule 3 |
| JUN | c-Jun |
| JUND | Jun-D |
| KCNH8 | Elk-3 |
| KIAA1271 | CARDIF |
| KIR2DS4 | KIR2DS4 |
| KIR3DL2 | KIR3DL2 |
| KIR3DL3 | KIR3DL3 |
| KITLG | Kit ligand |
| KLRB1 | CD161 |
| KLRC1 | NKG2A |
| KLRC2 | NKG2C |
| KLRC4 | NKG2F |
| KLRD1 | CD94 |
| KLRK1 | NKG2D |
| KPNA1 | karyopherin alpha 1 |
| | (importin alpha 5) |
| KRAS | Ras |
| KSR1 | KSR |
| LAG3 | LAG-3 |
| LAIR1 | LAIR1 |
| LAT | LAT |
| LBP | LBP |
| LCK | LCK |
| LCP2 | SLP-76 |
| LECT2 | leukocyte cell-derived |
| | chemotaxin 2 |
| LIF | leukemia inhibitory factor |
| LIFR | leukemia inhibitory factor |
| | receptor |
| LILRA1 | CD85i, LIR-6 |
| LILRA2 | CD85h, ILT1 |
| LILRA3 | CD85c, ILT6 |
| LILRA4 | CD85g, ILT7 |
| LILRA5 | CD85f, ILT11 |
| LILRA6 | CD85b, ILT8 |
| LILRB1 | CD85j, ILT2 |
| LILRB2 | CD85d, ILT4 |
| LILRB3 | CD85a, ILT5 |
| LILRB4 | CD85k, ILT3 |
| LILRB5 | CD85K, ILT3 |
| LILRP2 | CD85m, ILT10 |
| LIMS1 | PINCH1 |
| LMNA | Lamin A |
| LRRC23 | LRPB7 |
| LTA | lymphotoxin-alpha |
| LTA4H | LTA4H |
| LTBR | lymphotoxin-beta receptor |
| LTC4S | LTC4S |
| LY96 | MD-2 |
| LYN | Lyn |
| MADD | MADD |
| MAL | MAL |
| MALT1 | MALT1 |
| MAP2K1 | MEK1/2 |
| MAP2K2 | MEK1/2 |
| MAP2K3 | MKK3 |
| MAP2K4 | MKK4 |
| MAP2K6 | MKK6 |
| MAP2K7 | MKK7 |
| MAP3K1 | MEKK1 |
| MAP3K14 | NIK |
| MAP3K3 | MEKK3 |
| MAP3K5 | ASK1 |
| MAP3K6 | ASK2, MAP3K6 |
| MAP3K7 | TAK1 |
| MAP3K7IP1 | TAB1 |
| MAP3K7IP2 | TAB2 |
| MAP3K8 | Cot |
| MAP4K1 | HPK1, hematopoietic |
| | progenitor kinase 1 |
| MAPK1 | ERK1 |
| MAPK11 | p38 MAPK beta |
| MAPK12 | p38 MAPK gamma |
| MAPK13 | p38 MAPK delta |
| MAPK14 | p38 MAPK alpha |
| MAPK3 | ERK2 |
| MAPK8 | JNK1 |
| MAPK9 | MAPK9 |
| MAPKAPK2 | MAPKAPK2 |
| MAPKAPK3 | MAPKAPK3 |
| MAPKAPK5 | PRAK |
| MARCO | MARCO |
| MASP1 | MASP1 |
| MASP2 | MASP2 |
| MAX | Max |
| MBL2 | MBL |
| MCL1 | Mcl1 |
| MDM2 | MDM2 |
| MEF2A | MEF2A |
| MEF2B | MEF2B |
| MEF2C | MEF2C |
| MEF2D | MEF2D |
| MEFV | MEFV |
| MENA | ENAH |
| MGST2 | microsomal glutathione S- |
| | transferase 2 |
| MGST3 | microsomal glutathione S- |
| | transferase 3 |
| MICA | MIC-A |
| MICB | MIC-B |
| MIF | MIF |
| MKNK1 | MNK1 |
| MLCK | MLCK |
| MMP1 | MMP1 |
| MMP10 | MMP10 |
| MMP12 | MMP12 |
| MMP14 | MMP14 |
| MMP19 | MMP19 |
| MMP2 | MMP2 |
| MMP7 | MMP7 |
| MMP9 | MMP9 |
| MS4A1 | CD20 |
| MS4A2 | IgE receptor I, beta |
| | subunit |
| MSR1 | macrophage scavenger |
| | receptor 1 |
| MST1R | macrophage stimulating 1 |
| | receptor, CDw136 |
| MUC1 | CD227, mucin-1 |
| MYC | c-Myc |
| MYCN | N-Myc |
| MYD88 | MYD88 |
| MYH10 | myosin lib |
| MYH4 | beta-catenin |
| MYH9 | myosin lia |
| MYL6 | myosin, light polypeptide 6 |
| NALP1 | CARD7 |
| NALP12 | NALP12 |
| NALP2 | NALP2 |
| NALP6 | NALP6 |
| NCAM1 | CD56 |
| NCF2 | neutrophil cytosolic factor 2 |
| NCOA1 | NCOA1 |
| NCOA2 | nuclear receptor |
| | coactivator 2 |
| NCR1 | NKP46 |
| NCR2 | NKP44 |
| NCR3 | NKP30 |
| NFAT5 | NFAT5 |
| NFATC1 | NFATC1 |
| NFATC2 | NFATC2 |
| NFATC3 | NFATC3 |
| NFATC4 | NFATC4 |
| NFIL3 | NF-IL-3 |
| NFKB1 | NF-kB p105 |
| NFKB2 | NF-kB 2 |
| NFKBIA | IkappaB alpha |
| NFKBIB | IkappaB beta |
| NFKBIE | IkappaB-epsilon |
| NFRKB | NFRKB |
| NFX1 | NFX1 |
| NMI | NMI |
| NOD2 (CARD15) | nucleotide-binding |
| | oligomerization domain |
| | containing 2 (NOD2) |
| NOS2A | INOS |
| NOS3 | eNOS |
| NR2F1 | NR2F1 |
| NR3C1 | nuclear receptor 3C, |
| | glucocorticoid receptor |
| NR4A1 | Nuclear receptor 4A1 |
| NR4A2 | Nuclear receptor 4A2 |
| NRAS | Ras |
| NRIP1 | NRIP1 |
| NT5E | CD73 |
| OAS1 | OAS1 |
| OAS2 | OAS2 |
| OPRD1 | delta opioid receptor |
| | (DOR) |
| OPRK1 | kappa opioid receptor |
| | (KOR) |
| OPRM1 | mu opioid receptor (MOR) |
| OSM | Oncostatin M |
| OSMR | Oncostatin M receptor |
| PAG1 | PAG |
| PAK1 | PAK |
| PARP1 | PARP |
| PAX5 | PAX5 |
| PBEF1 | Pre-B cell enhancing |
| | factor |
| PDCD1LG2 | B7-DC |
| PDE1A | phosphodiesterase 1A |
| PDE1B | phosphodiesterase 1B |
| PDE1C | phosphodiesterase 1C |
| PDE2A | phosphodiesterase 2A |
| PDE3A | phosphodiesterase 3A |
| PDE3B | phosphodiesterase 3B |
| PDE4A | phosphodiesterase 4A |
| PDE4B | phosphodiesterase 4B |
| PDE4C | phosphodiesterase 4C |
| PDE4D | phosphodiesterase 4D |
| PDGFB | Platelet-derived growth |
| | factor B |
| PDGFRA | Platelet-derived growth |
| | factor receptor A |
| PDGFRB | Platelet-derived growth |
| | factor receptor B |
| PDPK1 | PDK1 |
| PECAM1 | PECAM1 |
| PFC | Factor P |
| PGDS | PGDS |
| PGLYRP1 | PGLYRP1 |
| PGLYRP2 | Peptidoglycan recognition |
| | protein 2 |
| PGLYRP3 | Peptidoglycan recognition |
| | protein 3 |
| PGLYRP4 | Peptidoglycan recognition |
| | protein 4 |
| PIAS1 | PIAS1 |
| PIAS2 | PIAS2 |
| PIAS3 | PIAS3 |
| PIAS4 | PIAS4 |
| PIGR | Poly-Ig receptor |
| PIK3AP1 | PI3KAP1, BCAP |
| PIK3CA | PI3K p110 |
| PIK3CB | PI3K p110 |
| PIK3CD | PI3K p110 |
| PIK3R1 | PI3K p85 |
| PIK3R2 | PI3K p85 |
| PIK3R3 | PI3K p85 |
| PIK3R5 | PI3K p101 |
| PILRB | Paired immunoglobulin- |
| | like receptor B |
| PLA2G2A | PLA3 |
| PLA2G2D | phospholipase A2 G2D |
| PLA2G4A | cPLA2 |
| PLAUR | CD87 |
| PLCB1 | phospholipase B1 |
| PLCB2 | phospholipase B2 |
| PLCB3 | phospholipase B3 |
| PLCB4 | phospholipase B4 |
| PLCG1 | PLC |
| PPARA | Peroxisome proliferator- |
| | activated receptor alpha |
| | (PPAR-alpha), or nuclear |
| | receptor subfamily 1, |
| | group C, member 1 |
| | (NR1C1) |
| PPARBP | PPAR binding protein |
| PPARG | PPAR |
| PPARGC1A | PPARGC1A |
| PPBP | CXC chemokine ligand 7 |
| PPM1A | protein phosphatase 1A, |
| | Mg2+ |
| PPP1CA | PP1 |
| PPP1CB | PP1 |
| PPP1CC | PP1 |
| PPP1R7 | PP1/PP2A |
| PPP2CA | PP2 |
| PPP2CB | PP2 |
| PPP2R1A | PP2 |
| PPP2R1B | protein phosphatase 2 R1 |
| | beta |
| PPP2R2B | protein phosphatase 2 R2 |
| | beta |
| PPP2R3A | PP1/PP2A |
| PPP3CA | Calcineurin |
| PPP3CB | Calcineurin |
| PPP3CC | Calcineurin |
| PPP3R1 | Calcineurin |
| PPP3R2 | Calcineurin |
| PRDX1 | peroxiredoxin 1 |
| PRDX2 | peroxiredoxin 2 |
| PRDX4 | peroxiredoxin 4 |
| PRF1 | Perforin-1 |
| PRG2 | proteoglycan 2 |
| PRKACA | PKA catalytic subunit |
| | alpha |
| PRKACB | PKA catalytic subunit beta |
| PRKACG | PKA catalytic subunit |
| | gamma |
| PRKAR1A | PKA regulatory subunit 1 |
| | alpha |
| PRKAR2A | PKA regulatory subunit 2 |
| | alpha |
| PRKAR2B | PKA regulatory subunit 2 |
| | beta |
| PRKCA | PKCalpha |
| PRKCB1 | PKCbeta |
| PRKCD | PKCdelta |
| PRKCE | PKCepsilon |
| PRKCQ | PKCtheta |
| PRKCZ | PKCaeta |
| PRKDC | Protein kinase, DNA- |
| | activated, catalytic |
| | polypeptide1 |
| PRKRA | PRKRA |
| PRSS16 | protease, serine, 16 |
| PRTN3 | Proteinase 3 |
| PSMA1 | PSMA1 |
| PSMB5 | PSMB5 |
| PSMB9 | PSMB9 |
| PSME1 | PSME1 |
| PSME2 | PSME2 |
| PTAFR | platelet-activating factor |
| | receptor |
| PTEN | PTEN |
| PTGDR | PTGDR |
| PTGDS | PGDS |
| PTGER1 | PTGER1 |
| PTGER2 | PTGER2 |
| PTGER3 | PTGER3 |
| PTGER4 | PTGER4 |
| PTGES | PGES |
| PTGES2 | PGES |
| PTGES3 | prostaglandin E synthase 3 |
| PTGFR | PTGFR |
| PTGIR | PTGIR |
| PTGIS | PGIS |
| PTK2 | FAK |
| PTK2B | PYK2 |
| PTPN1 | PTP1B |
| PTPN11 | SHP2 |
| PTPN13 | Fas-associated |
| | phosphatase-1 |
| PTPN2 | TC-PTP |
| PTPN22 | Protein tyrosine |
| | phosphatase, non- |
| | receptor type 22 |
| | (lymphoid), (PTPN22) |
| PTPN6 | SHP1 |
| PTPN7 | PTPN7 |
| PTPNS1 | PTPNS1 |
| PTP-PEST | PTPN12 |
| PTPRC | CD45 |
| PTPRJ | PTPRJ |
| PTPRK | Protein tyrosine |
| | phosphatase receptor type K |
| PTPRU | Protein tyrosine |
| | phosphatase receptor type U |
| PTX3 | pentraxin-3, TNFAIP5 |
| PXN | Paxillin |
| PYCARD | ASC/CARD5 |
| RAC1 | Rac |
| RAC2 | Rac |
| RAET1E | ULBP4 |
| RAF1 | c-Raf |
| RAG1 | Rag-1 |
| RAG2 | Rag-2 |
| RAP1A | Rap1a |
| RAP1GAP | Rap1GAP |
| RAPGEF1 | C3G |
| RAPGEF3 | EPAC |
| RASA1 | Ras GAP |
| RASGRP1 | Ras GRP |
| RASSF5 | RAPL |
| REL | c-REL |
| RELA | NF-kB p65 |
| RELB | RelB |
| RFX1 | regulatory factor X, 1 |
| RFX4 | regulatory factor X, 4 |
| RFXANK | regulatory factor X- |
| | associated ankyrin- |
| | containing protein |
| RGS1 | RGS1 |
| RHEB | Rheb |
| RHOA | RhoA |
| RHOH | RhoH |
| RIPK1 | RIP |
| RIPK2 | RIPK2 |
| RIPK3 | RIPK3 |
| ROCK1 | ROCK1 |
| ROCK2 | ROCK2 |
| RPS6KA1 | p90RSK |
| RPS6KA4 | MSK1 |
| RPS6KA5 | MSK2 |
| RPS6KB1 | p70 S6K |
| RPS6KB2 | p70 S6K |
| S100A12 | S100 A12 |
| S100A8 | S100 A8 |
| S100A9 | S100 A9 |
| SARM1 | SARM1 |
| SCARF1 | scavenger receptor class |
| | F, member 1 |
| SCARF2 | scavenger receptor class |
| | F, member 2 |
| SCGB1A1 | Unteroglobulin |
| SCGB3A1 | secretoglobin 3A1 |
| SCN9A | sodium channel, voltage- |
| | gated, type X, alpha |
| | (SCN9A) |
| SDPR | serum deprivation |
| | response |
| | (phosphatidylserine |
| | binding protein) |
| SECTM1 | secreted and |
| | transmembrane 1 |
| SELE | E-selectin |
| SELL | L-selectin, CD62L |
| SELP | P-selectin, CD62P |
| SELPLG | P-selectin Ligand |
| SEMA4D | CD100 |
| SERPINA1 | SERPINA1 |
| SERPINA5 | SERPINA5 |
| SERPINC1 | SERPINC1 |
| SERPIND1 | SERPIND1 |
| SERPINE1 | SERPINE1 |
| SERPINF2 | SERPINF2 |
| SERPING1 | SERPING1 |
| SGK | serum/glucocorticoid |
| | regulated kinase |
| SH2B1 | SH2-B PH domain |
| | containing signaling |
| | mediator 1 (SH2BPSM1) |
| SH2D1A | SAP |
| SH2D1B | EAT-2 |
| SH3BP2 | 3BP2 |
| SHB | SHB |
| SHC1 | SHC |
| SIGLEC1 | CD169, Siglec-1 |
| SIGLEC10 | SIGLEC10 |
| SIGLEC5 | Siglec-5, CD170 |
| SIGLEC7 | AIRM1 |
| SIPA1 | SIPA1 |
| SIRPB1 | CD172b |
| SIRPG | CD172g |
| SITPEC | SITPEC |
| SLA2 | Src-like adapter protein-2 |
| SLAMF1 | SLAMF1 |
| SLAMF6 | SLAMF6 |
| SLAMF7 | SLAMF7 |
| SLAMF9 | SLAMF9 |
| SLC22A1 (OCT1) | Solute carrier family 22 |
| | member 1 (SLC22A1) |
| SLC3A2 | CD98 |
| SLC7A5 | CD98 |
| SOCS1 | SOCS1 |
| SOCS2 | SOCS2 |
| SOCS3 | SOCS3 |
| SOCS4 | SOCS4 |
| SOCS5 | SOCS5 |
| SOCS6 | SOCS6 |
| SOD1 | SOD1 |
| SOD2 | SOD2 |
| SOS1 | SOS |
| SOS2 | SOS |
| SPN | CD43, leukosialin |
| SPTAN1 | Fodrin |
| SRC | Src |
| STAT1 | STAT1 |
| STAT2 | STAT2 |
| STAT3 | STAT3 |
| STAT4 | STAT4 |
| STAT5A | STAT5A |
| STAT5B | STAT5B |
| STAT6 | STAT6 |
| SYK | Syk |
| SYNGAP1 | Syn GAP |
| TACR1 | Tachykinin receptor 1 |
| TANK | TANK |
| TAP1 | TAP1 |
| TAP2 | TAP2 |
| TAPBP | TAP binding protein |
| TBK1 | NAK |
| TBX21 | T-box transcription factor |
| | (TBX21) |
| TBXA2R | TBXA2R |
| TBXAS1 | TXS |
| TCF7 | Transcription factor 7 |
| TCF8 | Transcription factor 8 |
| TCIRG1 | T-cell immune regulator 1 |
| TEC | Tec |
| TFEB | TFEB |
| TGFA | TGF-alpha |
| TGFB1 | TGF-beta1 |
| TGFB2 | TGF-beta2 |
| TGFB3 | TGF-beta3 |
| TGFBR1 | Type I Receptor |
| TGFBR2 | Type II Receptor |
| TGIF | TGIF |
| TGIF2 | TGFB-induced factor 2 |
| THEM4 | CTMP |
| THPO | thrombopoietin |
| THY1 | CD7 |
| TICAM1 | TICAM1, TRIF |
| TICAM2 | TICAM2 |
| TIRAP | TIRAP |
| TLN1 | Talin |
| TLN2 | Talin |
| TLR1 | TLR1 |
| TLR10 | TLR10 |
| TLR2 | TLR2 |
| TLR3 | TLR3 |
| TLR4 | TLR4 |
| TLR5 | TLR5 |
| TLR6 | TLR6 |
| TLR7 | TLR7 |
| TLR8 | TLR8 |
| TLR9 | TLR9 |
| TNFA | tumor necrosis factor- |
| | alpha (TNF-alpha) |
| TNFAIP3 | A20 |
| TNFAIP6 | TNFAIP6 |
| TNFRSF10A | DR4, TRAIL-R2 |
| TNFRSF10B | DR5, TRAIL-R1 |
| TNFRSF10C | DCR1, TRAIL-R3 |
| TNFRSF10D | DCR2, TRAIL-R4 |
| TNFRSF11A | TNFR4, TRANCE |
| TNFRSF11B | TNFRSF11B |
| TNFRSF12A | TNFRSF12A, TWEAK-R |
| TNFRSF13B | TNFRSF13B, CD267 |
| TNFRSF13C | TNFRSF13C, BAFF-R |
| TNFRSF14 | TNFRSF14, LIGHT-R |
| TNFRSF17 | TNFSF17, BCM, CD269 |
| TNFRSF19 | TNFRSF19 |
| TNFRSF1A | TNF-R1, CD120a |
| TNFRSF1B | TNF-R2, CD120b |
| TNFRSF21 | DR6 |
| TNFRSF25 | DR3, APO-3 |
| TNFRSF4 | TNFRSF4, OX40 |
| TNFRSF7 | CD27, TNFRSF7 |
| TNFRSF9 | TNFRSF9, 4-1BB |
| TNFSF10 | APO-2L, TRAIL |
| TNFSF11 | RANKL |
| TNFSF12 | APO-3L, TWEAK, DR3L |
| TNFSF13B | BAFF |
| TNFSF14 | TNFSF14, LIGHT, CD258 |
| TNFSF15 | TNFSF15, TL1A |
| TNFSF18 | TNFSF18 |
| TNFSF4 | TNFSF4, OX40L, CD252 |
| TNFSF7 | TNFSF7, CD70, CD27L |
| TNFSF8 | TNFSF8, CD153, CD30L |
| TNFSF9 | TNFSF9, 4-1BB-L |
| TNIP1 | TNFAIP3 interacting |
| | protein 1 |
| TOLLIP | TOLLIP |
| TP53 | p53 |
| TRADD | TRADD |
| TRAF1 | TRAF1 |
| TRAF2 | TRAF2 |
| TRAF3 | TRAF3 |
| TRAF5 | TRAF5 |
| TRAF6 | TRAF6 |
| TREM1 | TREM1 |
| TREM2 | TREM2 |
| TRGV9 | TCR gamma variable 9 |
| TSC1 | TSC1 |
| TSC2 | TSC2 |
| TTRAP | TTRAP |
| TXK | TXK tyrosine kinase |
| TXLNA | interleukin 14, taxilin alpha |
| TYK2 | TYK2 |
| TYROBP | DAP12 |
| UBE2N | UBE2N |
| UBE2V1 | UBE2V1 |
| ULBP3 | ULBP3 |
| VASP | VASP |
| VAV1 | Vav |
| VCAM1 | VCAM1 |
| VCL | vinculin |
| VEGF | VEGF |
| VIL2 | villin 2 (ezrin) |
| VPREB1 | vPreB |
| VTCN1 | B7-H4 |
| XBP1 | X-box binding protein 1 |
| XCL1 | Lymphotactin |
| XCR1 | XCR1 |
| YWHAB | 14-3-3beta |
| YWHAG | 14-3-3gamma |
| YWHAH | 14-3-3theta |
| YWHAQ | 14-3-3eta |
| YWHAZ | 14-3-3zeta |
| ZAP70 | Zap70 |

The identity of the inflammation-related protein whose chromosomal sequence is edited can and will vary. In preferred embodiments, the inflammation-related proteins whose chromosomal sequence is edited may be the monocyte chemoattractant protein-1 (MCP1) encoded by the Ccr2 gene, the C-C chemokine receptor type 5 (CCR5) encoded by the Ccr5 gene, the IgG receptor IIB (FCGR2b, also termed CD32) encoded by the Fcgr2b gene, the Fc epsilon R1g (FCER1g) protein encoded by the Fcerlg gene, the forkhead box N1 transcription factor (FOXN1) encoded by the FOXN1 gene, Interferon-gamma (IFN-γ) encoded by the IFNg gene, interleukin 4 (IL-4) encoded by the IL-4 gene, perforin-1 encoded by the PRF-1 gene, the cyclooxygenase 1 protein (COX1) encoded by the COX1 gene, the cyclooxygenase 2 protein (COX2) encoded by the COX2 gene, the T-box transcription factor (TBX21) protein encoded by the TBX21 gene, the SH2-B PH domain containing signaling mediator 1 protein (SH2BPSM1) encoded by the SH2B1 gene (also termed SH2BPSM1), the fibroblast growth factor receptor 2 (FGFR2) protein encoded by the FGFR2 gene, the solute carrier family 22 member 1 (SLC22A1) protein encoded by the OCT1 gene (also termed SLC22A1), the peroxisome proliferator-activated receptor alpha protein (PPAR-alpha, also termed the nuclear receptor subfamily 1, group C, member 1; NR1C1) encoded by the PPARA gene, the phosphatase and tensin homolog protein (PTEN) encoded by the PTEN gene, interleukin 1 alpha (IL-1α) encoded by the IL-1A gene, interleukin 1 beta (IL-1β) encoded by the IL-1B gene, interleukin 6 (IL-6) encoded by the IL-6 gene, interleukin 10 (IL-10) encoded by the IL-10 gene, interleukin 12 alpha (IL-12α) encoded by the IL-12A gene, interleukin 12 beta (IL-12β) encoded by the IL-12B gene, interleukin 13 (IL-13) encoded by the IL-13 gene, interleukin 17A(IL-17A, also termed CTLA8) encoded by the IL-17A gene, interleukin 17B(IL-17B) encoded by the IL-17B gene, interleukin 17C (IL-17C) encoded by the IL-17C gene interleukin 17D (IL-17D) encoded by the IL-17D gene interleukin 17F (IL-17F) encoded by the IL-17F gene, interleukin 23 (IL-23) encoded by the IL-23 gene, the chemokine (C-X3-C motif) receptor 1 protein (CX3CR1) encoded by the CX3CR1 gene, the chemokine (C-X3-C motif) ligand 1 protein (CX3CL1) encoded by the CX3CL1 gene, the recombination activating gene 1 protein (RAG1) encoded by the RAG1 gene, the recombination activating gene 2 protein (RAG2) encoded by the RAG2 gene, the protein kinase, DNA-activated, catalytic polypeptide1 (PRKDC) encoded by the PRKDC (DNAPK) gene, the protein tyrosine phosphatase non-receptor type 22 protein (PTPN22) encoded by the PTPN22 gene, tumor necrosis factor alpha (TNFα) encoded by the TNFA gene, the nucleotide-binding oligomerization domain containing 2 protein (NOD2) encoded by the NOD2 gene (also termed CARD15), or the cytotoxic T-lymphocyte antigen 4 protein (CTLA4, also termed CD152) encoded by the CTLA4 gene. In an exemplary embodiment, the genetically modified animal is a rat, and the edited chromosomal sequence encoding the inflammation-related protein is as listed in Table B.

TABLE B

| Edited Chromosomal | | NCBI Reference |
|---|---|---|
| Sequence | Encoded Protein | Sequence |
| Ccr2 | monocyte chemoattractant | NM_021866 |
| | protein-1 (MCP1) | |
| Ccr5 | C-C chemokine receptor type | NM_053960 |
| | 5 (CCR5) | |
| COX1 | cytochrome c oxidase 1 or | NM_017043 |
| | cyclooxygenase 1 (COX1) | |
| COX2 | cytochrome c oxidase 2 | NM_017232 |
| | (COX2) | |
| CTLA4 | Cytotoxic T-Lymphocyte | NM_031674 |
| | Antigen 4 (CTLA4, CD152) | |
| CX3CL1 | Chemokine (C—X3—C | NM_134455 |
| | motif) ligand 1 (CX3CL1) | |
| CX3CR1 | Chemokine (C—X3—C | NM_133534 |
| | motif) receptor 1 (CX3CR1) | |
| FCER1G | FCER1g (Fc epsilon R1g) | NM_00131001 |
| FCGR2B | IgG receptor IIB, (FCGR2b or | NM_175756 |
| | CD32) | |
| FGFR2 | Fibroblast growth factor | NW_001084773 |
| | receptor 2 (FGFR2) | |
| IFNG | Interferon-gamma (IFN-) | NM_138880 |
| IL-10 | Interleukin-10 (IL-10 or | NM_012854 |
| | IL10), also known as human | |
| | cytokine synthesis inhibitory | |
| | factor (CSIF) | |
| IL-12A | Subunit alpha of interleukin | NM_053390 |
| | 12 | |
| IL-12B | Subunit beta of interleukin 12 | NM_022611 |
| IL-13 | Interleukin 13 (IL-13) | NM_053828 |
| IL17A | interleukin 17A | NM_001106897 |
| IL17B | interleukin 17B | NM_053789 |
| IL-17C | Interleukin 17C | XM_002725399, |
| | | XM_001078615 |
| IL-17D | Interleukin 17D | XM_001079675 |
| IL-17F | Interleukin 17F | NM_001015011 |
| IL1A | interleukin 1, alpha | NM_017019 |
| IL-1B | Interleukin-1 beta (IL-1beta) | NM_031512 |
| IL-23 | Interleukin 23 | NM_130410 |
| IL-4 | Interleukin-4 (IL-4) | NM_201270 |
| IL-6 | Interleukin-6 (IL-6) | NM_012589 |
| NOD2 (CARD15) | nucleotide-binding | NM_001106172 |
| | oligomerization domain | |
| | containing 2 (NOD2) | |
| PPARA | Peroxisome proliferator- | NM_013196 |
| | activated receptor alpha | |
| | (PPAR-alpha), or nuclear | |
| | receptor subfamily 1, group | |
| | C, member 1 (NR1C1) | |
| PRF1 | Perforin-1 | NM_{—} |
| PTEN | phosphatase and tensin | NM_031606 |
| | homolog (PTEN) | |
| PTPN22 | Protein tyrosine | NM_001106460 |
| | phosphatase, non-receptor | |
| | type 22 (lymphoid), | |
| | (PTPN22) | |
| RAG1 | recombination activating | XM_001079242 |
| | gene 1 (RAG1) | |
| SH2B1 | SH2-B PH domain containing | NM_001048180, |
| | signaling mediator 1 | NM_134456 |
| | (SH2BPSM1) | |
| SLC22A1 (OCT1) | Solute carrier family 22 | NM_012697 |
| | member 1 (SLC22A1) | |
| TBX21 | T-box transcription factor | NM_001107043 |
| | (TBX21) | |
| TNFA | tumor necrosis factor-alpha | NM_012675 |
| | (TNF-alpha) | |

The animal or cell may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more disrupted chromosomal sequences encoding an inflammation-related protein and zero, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chromosomally integrated sequences encoding the disrupted inflammation-related protein.

The edited or integrated chromosomal sequence may be modified to encode an altered inflammation-related protein. A number of mutations in inflammation-related chromosomal sequences have been associated with inflammation. For instance, the Delta 32 mutation in CCR5 results in the genetic deletion of the CCR5 gene, which plays a role in inflammatory responses to infection. Homozygous carriers of this mutation are resistant to HIV-1 infection. Missense and truncating mutations in perforin-1, such as W374stop (i.e. tryptophan at position 219 is changed to stop codon producing a truncated polypeptide), V50M (i.e. valine at position 50 is changed to a methionine) and I224D (i.e. isoleucine at position 224 is changed to aspartate), have been shown to cause familial hemophagocytic lymphohistiocytosis (FHL). Missense mutations or copy number gains of FGFR2 gene are associated with Crouzon syndrome, Pfeiffer syndrome, Craniosynostosis, Apert syndrome, Jackson-Weiss syndrome, Beare-Stevenson cutis gyrata syndrome, Saethre-Chotzen syndrome, and syndromic craniosynostosis. Other associations of genetic variants in inflammation-associated genes and disease are known are known in the art. See, for example, Loza et al. (2007) PLoS One 10:e1035, the disclosure of which is incorporated by reference herein in its entirety.

### (b) Animals

The term “animal,” as used herein, refers to a non-human animal. The animal may be an embryo, a juvenile, or an adult. Suitable animals include vertebrates such as mammals, birds, reptiles, amphibians, and fish. Examples of suitable mammals include without limit rodents, companion animals, livestock, and primates. Non-limiting examples of rodents include mice, rats, hamsters, gerbils, and guinea pigs. Suitable companion animals include but are not limited to cats, dogs, rabbits, hedgehogs, and ferrets. Non-limiting examples of livestock include horses, goats, sheep, swine, cattle, llamas, and alpacas. Suitable primates include but are not limited to capuchin monkeys, chimpanzees, lemurs, macaques, marmosets, tamarins, spider monkeys, squirrel monkeys, and vervet monkeys. Non-limiting examples of birds include chickens, turkeys, ducks, and geese. Alternatively, the animal may be an invertebrate such as an insect, a nematode, and the like. Non-limiting examples of insects include Drosophila and mosquitoes. An exemplary animal is a rat. Non-limiting examples of suitable rat strains include Dahl Salt-Sensitive, Fischer 344, Lewis, Long Evans Hooded, Sprague-Dawley, and Wistar. Non-limiting examples of commonly used rat strains suitable for genetic manipulation include Dahl Salt-Sensitive, Fischer 344, Lewis, Long Evans Hooded, Sprague-Dawley and Wistar. In another iteration of the invention, the animal does not comprise a genetically modified mouse. In each of the foregoing iterations of suitable animals for the invention, the animal does not include exogenously introduced, randomly integrated transposon sequences.

### (c) Inflammation-Related Proteins

The inflammation-related protein may be from any of the animals listed above. Furthermore, the inflammation-related protein may be a human inflammation-related protein. Additionally, the inflammation-related protein may be a bacterial, fungal, or plant protein. The type of animal and the source of the protein can and will vary. As an example, the genetically modified animal may be a rat, cat, dog, or pig, and the inflammation-related protein may be human. Alternatively, the genetically modified animal may be a rat, cat, or pig, and the inflammation-related protein may be canine. One of skill in the art will readily appreciate that numerous combinations are possible and are encompassed by the present invention. In an exemplary embodiment, the genetically modified animal is a rat, and the inflammation-related protein is human.

Additionally, the inflammation-related gene may be modified to include a tag or reporter gene or genes as are well-known. Reporter genes include those encoding selectable markers such as chloramphenicol acetyltransferase (CAT) and neomycin phosphotransferase (neo), and those encoding a fluorescent protein such as green fluorescent protein (GFP), red fluorescent protein, or any genetically engineered variant thereof that improves the reporter performance. Non-limiting examples of known such FP variants include EGFP, blue fluorescent protein (EBFP, EBFP2, Azurite, mKalama1), cyan fluorescent protein (ECFP, Cerulean, CyPet) and yellow fluorescent protein derivatives (YFP, Citrine, Venus, YPet). For example, in a genetic construct containing a reporter gene, the reporter gene sequence can be fused directly to the targeted gene to create a gene fusion. A reporter sequence can be integrated in a targeted manner in the targeted gene, for example the reporter sequences may be integrated specifically at the 5′ or 3′ end of the targeted gene. The two genes are thus under the control of the same promoter elements and are transcribed into a single messenger RNA molecule. Alternatively, the reporter gene may be used to monitor the activity of a promoter in a genetic construct, for example by placing the reporter sequence downstream of the target promoter such that expression of the reporter gene is under the control of the target promoter, and activity of the reporter gene can be directly and quantitatively measured, typically in comparison to activity observed under a strong consensus promoter. It will be understood that doing so may or may not lead to destruction of the targeted gene.

### (II) Genetically Modified Cells

A further aspect of the present disclosure provides genetically modified cells or cell lines comprising at least one edited chromosomal sequence encoding an inflammation-related protein. The genetically modified cell or cell line may be derived from any of the genetically modified animals disclosed herein. Alternatively, the chromosomal sequence coding an inflammation-related protein may be edited in a cell as detailed below. The disclosure also encompasses a lysate of said cells or cell lines.

In general, the cells will be eukaryotic cells. Suitable host cells include fungi or yeast, such as Pichia, Saccharomyces, or Schizosaccharomyces; insect cells, such as SF9 cells from Spodoptera frugiperda or S2 cells from Drosophila melanogaster; and animal cells, such as mouse, rat, hamster, non-human primate, or human cells. Exemplary cells are mammalian. The mammalian cells may be primary cells. In general, any primary cell that is sensitive to double strand breaks may be used. The cells may be of a variety of cell types, e.g., fibroblast, myoblast, T or B cell, macrophage, epithelial cell, and so forth.

When mammalian cell lines are used, the cell line may be any established cell line or a primary cell line that is not yet described. The cell line may be adherent or non-adherent, or the cell line may be grown under conditions that encourage adherent, non-adherent or organotypic growth using standard techniques known to individuals skilled in the art. Non-limiting examples of suitable mammalian cell lines include Chinese hamster ovary (CHO) cells, monkey kidney CVI line transformed by SV40 (COS7), human embryonic kidney line 293, baby hamster kidney cells (BHK), mouse sertoli cells (TM4), monkey kidney cells (CVI-76), African green monkey kidney cells (VERO), human cervical carcinoma cells (HeLa), canine kidney cells (MDCK), buffalo rat liver cells (BRL 3A), human lung cells (W138), human liver cells (Hep G2), mouse mammary tumor cells (MMT), rat hepatoma cells (HTC), HIH/3T3 cells, the human U2-OS osteosarcoma cell line, the human A549 cell line, the human K562 cell line, the human HEK293 cell lines, the human HEK293T cell line, and TRI cells. For an extensive list of mammalian cell lines, those of ordinary skill in the art may refer to the American Type Culture Collection catalog (ATCC®, Mamassas, Va.).

In still other embodiments, the cell may be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, and unipotent stem cells.

### (III) Zinc Finger-Mediated Genomic Editing

In general, the genetically modified animal or cell detailed above in sections (I) and (II), respectively, is generated using a zinc finger nuclease-mediated genome editing process. The process for editing a chromosomal sequence comprises: (a) introducing into an embryo or cell at least one nucleic acid encoding a zinc finger nuclease that recognizes a target sequence in the chromosomal sequence and is able to cleave a site in the chromosomal sequence, and, optionally, (i) at least one donor polynucleotide comprising a sequence for integration flanked by an upstream sequence and a downstream sequence that share substantial sequence identity with either side of the cleavage site, or (ii) at least one exchange polynucleotide comprising a sequence that is substantially identical to a portion of the chromosomal sequence at the cleavage site and which further comprises at least one nucleotide change; and (b) culturing the embryo or cell to allow expression of the zinc finger nuclease such that the zinc finger nuclease introduces a double-stranded break into the chromosomal sequence, and wherein the double-stranded break is repaired by (i) a non-homologous end-joining repair process such that an inactivating mutation is introduced into the chromosomal sequence, or (ii) a homology-directed repair process such that the sequence in the donor polynucleotide is integrated into the chromosomal sequence or the sequence in the exchange polynucleotide is exchanged with the portion of the chromosomal sequence.

Components of the zinc finger nuclease-mediated method are described in more detail below.

### (a) Zinc Finger Nuclease

The method comprises, in part, introducing into an embryo or cell at least one nucleic acid encoding a zinc finger nuclease. Typically, a zinc finger nuclease comprises a DNA binding domain (i.e., zinc finger) and a cleavage domain (i.e., nuclease). The DNA binding and cleavage domains are described below. The nucleic acid encoding a zinc finger nuclease may comprise DNA or RNA. For example, the nucleic acid encoding a zinc finger nuclease may comprise mRNA. When the nucleic acid encoding a zinc finger nuclease comprises mRNA, the mRNA molecule may be 5′ capped. Similarly, when the nucleic acid encoding a zinc finger nuclease comprises mRNA, the mRNA molecule may be polyadenylated. An exemplary nucleic acid according to the method is a capped and polyadenylated mRNA molecule encoding a zinc finger nuclease. Methods for capping and polyadenylating mRNA is known in the art.

(i) Zinc Finger Binding Domain

Zinc finger binding domains may be engineered to recognize and bind to any nucleic acid sequence of choice. See, for example, Beerli et al. (2002) Nat. Biotechnol. 20:135-141; Pabo et al. (2001) Ann. Rev. Biochem. 70:313-340; Isalan et al. (2001) Nat. Biotechnol. 19:656-660; Segal et al. (2001) Curr. Opin. Biotechnol. 12:632-637; Choo et al. (2000) Curr. Opin. Struct. Biol. 10:411-416; Zhang et al. (2000) J. Biol. Chem. 275(43):33850-33860; Doyon et al. (2008) Nat. Biotechnol. 26:702-708; and Santiago et al. (2008) Proc. Natl. Acad. Sci. USA 105:5809-5814. An engineered zinc finger binding domain may have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence. See, for example, U.S. Pat. Nos. 6,453,242 and 6,534,261, the disclosures of which are incorporated by reference herein in their entireties. As an example, the algorithm of described in U.S. Pat. No. 6,453,242 may be used to design a zinc finger binding domain to target a preselected sequence. Alternative methods, such as rational design using a nondegenerate recognition code table may also be used to design a zinc finger binding domain to target a specific sequence (Sera et al. (2002) Biochemistry 41:7074-7081). Publically available web-based tools for identifying potential target sites in DNA sequences and designing zinc finger binding domains may be found at http://www.zincfingertools.org and http://bindr.gdcb.iastate.edu/ZiFiT/, respectively (Mandell et al. (2006) Nuc. Acid Res. 34:W516-W523; Sander et al. (2007) Nuc. Acid Res. 35:W599-W605).

A zinc finger binding domain may be designed to recognize a DNA sequence ranging from about 3 nucleotides to about 21 nucleotides in length, or from about 8 to about 19 nucleotides in length. In general, the zinc finger binding domains of the zinc finger nucleases disclosed herein comprise at least three zinc finger recognition regions (i.e., zinc fingers). In one embodiment, the zinc finger binding domain may comprise four zinc finger recognition regions. In another embodiment, the zinc finger binding domain may comprise five zinc finger recognition regions. In still another embodiment, the zinc finger binding domain may comprise six zinc finger recognition regions. A zinc finger binding domain may be designed to bind to any suitable target DNA sequence. See for example, U.S. Pat. Nos. 6,607,882; 6,534,261 and 6,453,242, the disclosures of which are incorporated by reference herein in their entireties.

Exemplary methods of selecting a zinc finger recognition region may include phage display and two-hybrid systems, and are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237, each of which is incorporated by reference herein in its entirety. In addition, enhancement of binding specificity for zinc finger binding domains has been described, for example, in WO 02/077227.

Zinc finger binding domains and methods for design and construction of fusion proteins (and polynucleotides encoding same) are known to those of skill in the art and are described in detail in U.S. Patent Application Publication Nos. 20050064474 and 20060188987, each incorporated by reference herein in its entirety. Zinc finger recognition regions and/or multi-fingered zinc finger proteins may be linked together using suitable linker sequences, including for example, linkers of five or more amino acids in length. See, U.S. Pat. Nos. 6,479,626; 6,903,185; and 7,153,949, the disclosures of which are incorporated by reference herein in their entireties, for non-limiting examples of linker sequences of six or more amino acids in length. The zinc finger binding domain described herein may include a combination of suitable linkers between the individual zinc fingers of the protein.

In some embodiments, the zinc finger nuclease may further comprise a nuclear localization signal or sequence (NLS). A NLS is an amino acid sequence, which facilitates targeting the zinc finger nuclease protein into the nucleus to introduce a double stranded break at the target sequence in the chromosome. Nuclear localization signals are known in the art. See, for example, Makkerh et al. (1996) Current Biology 6:1025-1027.

(ii) Cleavage Domain

A zinc finger nuclease also includes a cleavage domain. The cleavage domain portion of the zinc finger nucleases disclosed herein may be obtained from any endonuclease or exonuclease. Non-limiting examples of endonucleases from which a cleavage domain may be derived include, but are not limited to, restriction endonucleases and homing endonucleases. See, for example, 2002-2003 Catalog, New England Biolabs, Beverly, Mass.; and Belfort et al. (1997) Nucleic Acids Res. 25:3379-3388 or www.neb.com. Additional enzymes that cleave DNA are known (e.g., S1 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) may be used as a source of cleavage domains.

A cleavage domain also may be derived from an enzyme or portion thereof, as described above, that requires dimerization for cleavage activity. Two zinc finger nucleases may be required for cleavage, as each nuclease comprises a monomer of the active enzyme dimer. Alternatively, a single zinc finger nuclease may comprise both monomers to create an active enzyme dimer. As used herein, an “active enzyme dimer” is an enzyme dimer capable of cleaving a nucleic acid molecule. The two cleavage monomers may be derived from the same endonuclease (or functional fragments thereof), or each monomer may be derived from a different endonuclease (or functional fragments thereof).

When two cleavage monomers are used to form an active enzyme dimer, the recognition sites for the two zinc finger nucleases are preferably disposed such that binding of the two zinc finger nucleases to their respective recognition sites places the cleavage monomers in a spatial orientation to each other that allows the cleavage monomers to form an active enzyme dimer, e.g., by dimerizing. As a result, the near edges of the recognition sites may be separated by about 5 to about 18 nucleotides. For instance, the near edges may be separated by about 5, 6, 7, 8, 9, 10, 11, 12, 13, 14, 15, 16, 17 or 18 nucleotides. It will however be understood that any integral number of nucleotides or nucleotide pairs may intervene between two recognition sites (e.g., from about 2 to about 50 nucleotide pairs or more). The near edges of the recognition sites of the zinc finger nucleases, such as for example those described in detail herein, may be separated by 6 nucleotides. In general, the site of cleavage lies between the recognition sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31, 978-31, 982. Thus, a zinc finger nuclease may comprise the cleavage domain from at least one Type IIS restriction enzyme and one or more zinc finger binding domains, which may or may not be engineered. Exemplary Type IIS restriction enzymes are described for example in International Publication WO 07/014,275, the disclosure of which is incorporated by reference herein in its entirety. Additional restriction enzymes also contain separable binding and cleavage domains, and these also are contemplated by the present disclosure. See, for example, Roberts et al. (2003) Nucleic Acids Res. 31:418-420.

An exemplary Type IIS restriction enzyme, whose cleavage domain is separable from the binding domain, is Fok I. This particular enzyme is active as a dimmer (Bitinaite et al. (1998) Proc. Natl. Acad. Sci. USA 95: 10, 570-10, 575). Accordingly, for the purposes of the present disclosure, the portion of the Fok I enzyme used in a zinc finger nuclease is considered a cleavage monomer. Thus, for targeted double-stranded cleavage using a Fok I cleavage domain, two zinc finger nucleases, each comprising a FokI cleavage monomer, may be used to reconstitute an active enzyme dimer. Alternatively, a single polypeptide molecule containing a zinc finger binding domain and two Fok I cleavage monomers may also be used.

In certain embodiments, the cleavage domain may comprise one or more engineered cleavage monomers that minimize or prevent homodimerization, as described, for example, in U.S. Patent Publication Nos. 20050064474, 20060188987, and 20080131962, each of which is incorporated by reference herein in its entirety. By way of non-limiting example, amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains. Exemplary engineered cleavage monomers of Fok I that form obligate heterodimers include a pair in which a first cleavage monomer includes mutations at amino acid residue positions 490 and 538 of Fok I and a second cleavage monomer that includes mutations at amino-acid residue positions 486 and 499.

Thus, in one embodiment, a mutation at amino acid position 490 replaces Glu (E) with Lys (K); a mutation at amino acid residue 538 replaces Iso (I) with Lys (K); a mutation at amino acid residue 486 replaces Gln (Q) with Glu (E); and a mutation at position 499 replaces Iso (I) with Lys (K). Specifically, the engineered cleavage monomers may be prepared by mutating positions 490 from E to K and 538 from Ito K in one cleavage monomer to produce an engineered cleavage monomer designated “E490K:I538K” and by mutating positions 486 from Q to E and 499 from Ito L in another cleavage monomer to produce an engineered cleavage monomer designated “Q486E:I499L.” The above described engineered cleavage monomers are obligate heterodimer mutants in which aberrant cleavage is minimized or abolished. Engineered cleavage monomers may be prepared using a suitable method, for example, by site-directed mutagenesis of wild-type cleavage monomers (Fok I) as described in U.S. Patent Publication No. 20050064474 (see Example 5).

The zinc finger nuclease described above may be engineered to introduce a double stranded break at the targeted site of integration. The double stranded break may be at the targeted site of integration, or it may be up to 1, 2, 3, 4, 5, 10, 15, 20, 25, 30, 35, 40, 45, 50, 100, or 1000 nucleotides away from the site of integration. In some embodiments, the double stranded break may be up to 1, 2, 3, 4, 5, 10, 15, or 20 nucleotides away from the site of integration. In other embodiments, the double stranded break may be up to 10, 15, 20, 25, 30, 35, 40, 45, or 50 nucleotides away from the site of integration. In yet other embodiments, the double stranded break may be up to 50, 100, or 1000 nucleotides away from the site of integration.

### (b) Optional Donor Polynucleotide

The method for editing chromosomal sequences encoding inflammation-related proteins may further comprise introducing at least one donor polynucleotide comprising a sequence encoding an inflammation-related protein into the embryo or cell. A donor polynucleotide comprises at least three components: the sequence coding the inflammation-related protein, an upstream sequence, and a downstream sequence. The sequence encoding the protein is flanked by the upstream and downstream sequence, wherein the upstream and downstream sequences share sequence similarity with either side of the site of integration in the chromosome.

Typically, the donor polynucleotide will be DNA. The donor polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary donor polynucleotide comprising the sequence encoding the inflammation-related protein may be a BAC.

The sequence of the donor polynucleotide that encodes the inflammation-related protein may include coding (i.e., exon) sequence, as well as intron sequences and upstream regulatory sequences (such as, e.g., a promoter). Depending upon the identity and the source of the sequence encoding the inflammation-related protein, the size of the sequence encoding the inflammation-related protein will vary. For example, the sequence encoding the inflammation-related protein may range in size from about 1 kb to about 5,000 kb.

The donor polynucleotide also comprises upstream and downstream sequence flanking the sequence encoding the inflammation-related protein. The upstream and downstream sequences in the donor polynucleotide are selected to promote recombination between the chromosomal sequence of interest and the donor polynucleotide. The upstream sequence, as used herein, refers to a nucleic acid sequence that shares sequence similarity with the chromosomal sequence upstream of the targeted site of integration. Similarly, the downstream sequence refers to a nucleic acid sequence that shares sequence similarity with the chromosomal sequence downstream of the targeted site of integration. The upstream and downstream sequences in the donor polynucleotide may share about 75%, 80%, 85%, 90%, 95%, or 100% sequence identity with the targeted chromosomal sequence. In other embodiments, the upstream and downstream sequences in the donor polynucleotide may share about 95%, 96%, 97%, 98%, 99%, or 100% sequence identity with the targeted chromosomal sequence. In an exemplary embodiment, the upstream and downstream sequences in the donor polynucleotide may share about 99% or 100% sequence identity with the targeted chromosomal sequence.

An upstream or downstream sequence may comprise from about 50 bp to about 2500 bp. In one embodiment, an upstream or downstream sequence may comprise about 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. An exemplary upstream or downstream sequence may comprise about 200 bp to about 2000 bp, about 600 bp to about 1000 bp, or more particularly about 700 bp to about 1000 bp.

In some embodiments, the donor polynucleotide may further comprise a marker. Such a marker may make it easy to screen for targeted integrations. Non-limiting examples of suitable markers include restriction sites, fluorescent proteins, or selectable markers.

One of skill in the art would be able to construct a donor polynucleotide as described herein using well-known standard recombinant techniques (see, for example, Sambrook et al., 2001 and Ausubel et al., 1996).

In the method detailed above for integrating a sequence encoding the inflammation-related protein, a double stranded break introduced into the chromosomal sequence by the zinc finger nuclease is repaired, via homologous recombination with the donor polynucleotide, such that the sequence encoding the inflammation-related protein is integrated into the chromosome. The presence of a double-stranded break facilitates integration of the sequence encoding the inflammation-related protein. A donor polynucleotide may be physically integrated or, alternatively, the donor polynucleotide may be used as a template for repair of the break, resulting in the introduction of the sequence encoding the inflammation-related protein as well as all or part of the upstream and downstream sequences of the donor polynucleotide into the chromosome. Thus, endogenous chromosomal sequence may be converted to the sequence of the donor polynucleotide.

### (c) Optional Exchange Polynucleotide

The method for editing chromosomal sequences encoding an inflammation-related protein may further comprise introducing into the embryo or cell at least one exchange polynucleotide comprising a sequence that is substantially identical to the chromosomal sequence at the site of cleavage and which further comprises at least one specific nucleotide change.

Typically, the exchange polynucleotide will be DNA. The exchange polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary exchange polynucleotide may be a DNA plasmid.

The sequence in the exchange polynucleotide is substantially identical to a portion of the chromosomal sequence at the site of cleavage. In general, the sequence of the exchange polynucleotide will share enough sequence identity with the chromosomal sequence such that the two sequences may be exchanged by homologous recombination. For example, the sequence in the exchange polynucleotide may have at least about 80, 81, 82, 83, 84, 85, 86, 87, 88, 89, 90, 91, 92, 93, 94, 95, 96, 97, 98, or 99% sequence identity with a portion of the chromosomal sequence.

Importantly, the sequence in the exchange polynucleotide comprises at least one specific nucleotide change with respect to the sequence of the corresponding chromosomal sequence. For example, one nucleotide in a specific codon may be changed to another nucleotide such that the codon codes for a different amino acid. In one embodiment, the sequence in the exchange polynucleotide may comprise one specific nucleotide change such that the encoded protein comprises one amino acid change. In other embodiments, the sequence in the exchange polynucleotide may comprise two, three, four, or more specific nucleotide changes such that the encoded protein comprises one, two, three, four, or more amino acid changes. In still other embodiments, the sequence in the exchange polynucleotide may comprise a three nucleotide deletion or insertion such that the reading frame of the coding reading is not altered (and a functional protein is produced). The expressed protein, however, would comprise a single amino acid deletion or insertion.

The length of the sequence in the exchange polynucleotide that is substantially identical to a portion of the chromosomal sequence at the site of cleavage can and will vary. In general, the sequence in the exchange polynucleotide may range from about 50 bp to about 10,000 bp in length. In various embodiments, the sequence in the exchange polynucleotide may be about 100, 200, 400, 600, 800, 1000, 1200, 1400, 1600, 1800, 2000, 2200, 2400, 2600, 2800, 3000, 3200, 3400, 3600, 3800, 4000, 4200, 4400, 4600, 4800, or 5000 bp in length. In other embodiments, the sequence in the exchange polynucleotide may be about 5500, 6000, 6500, 6000, 6500, 7000, 7500, 8000, 8500, 9000, 9500, or 10,000 bp in length.

One of skill in the art would be able to construct an exchange polynucleotide as described herein using well-known standard recombinant techniques (see, for example, Sambrook et al., 2001 and Ausubel et al., 1996).

In the method detailed above for modifying a chromosomal sequence, a double stranded break introduced into the chromosomal sequence by the zinc finger nuclease is repaired, via homologous recombination with the exchange polynucleotide, such that the sequence in the exchange polynucleotide may be exchanged with a portion of the chromosomal sequence. The presence of the double stranded break facilitates homologous recombination and repair of the break. The exchange polynucleotide may be physically integrated or, alternatively, the exchange polynucleotide may be used as a template for repair of the break, resulting in the exchange of the sequence information in the exchange polynucleotide with the sequence information in that portion of the chromosomal sequence. Thus, a portion of the endogenous chromosomal sequence may be converted to the sequence of the exchange polynucleotide. The changed nucleotide(s) may be at or near the site of cleavage. Alternatively, the changed nucleotide(s) may be anywhere in the exchanged sequences. As a consequence of the exchange, however, the chromosomal sequence is modified.

### (d) Delivery of Nucleic Acids

To mediate zinc finger nuclease genomic editing, at least one nucleic acid molecule encoding a zinc finger nuclease and, optionally, at least one exchange polynucleotide or at least one donor polynucleotide are delivered to the embryo or the cell of interest. Typically, the embryo is a fertilized one-cell stage embryo of the species of interest.

Suitable methods of introducing the nucleic acids to the embryo or cell include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In one embodiment, the nucleic acids may be introduced into an embryo by microinjection. The nucleic acids may be microinjected into the nucleus or the cytoplasm of the embryo. In another embodiment, the nucleic acids may be introduced into a cell by nucleofection.

In embodiments in which both a nucleic acid encoding a zinc finger nuclease and a donor (or exchange) polynucleotide are introduced into an embryo or cell, the ratio of donor (or exchange) polynucleotide to nucleic acid encoding a zinc finger nuclease may range from about 1:10 to about 10:1. In various embodiments, the ratio of donor (or exchange) polynucleotide to nucleic acid encoding a zinc finger nuclease may be about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In one embodiment, the ratio may be about 1:1.

In embodiments in which more than one nucleic acid encoding a zinc finger nuclease and, optionally, more than one donor (or exchange) polynucleotide are introduced into an embryo or cell, the nucleic acids may be introduced simultaneously or sequentially. For example, nucleic acids encoding the zinc finger nucleases, each specific for a distinct recognition sequence, as well as the optional donor (or exchange) polynucleotides, may be introduced at the same time. Alternatively, each nucleic acid encoding a zinc finger nuclease, as well as the optional donor (or exchange) polynucleotides, may be introduced sequentially.

### (e) Culturing the Embryo or Cell

The method of inducing genomic editing with a zinc finger nuclease further comprises culturing the embryo or cell comprising the introduced nucleic acid(s) to allow expression of the zinc finger nuclease. An embryo may be cultured in vitro (e.g., in cell culture). Typically, the embryo is cultured at an appropriate temperature and in appropriate media with the necessary O₂/CO₂ ratio to allow the expression of the zinc finger nuclease. Suitable non-limiting examples of media include M2, M16, KSOM, BMOC, and HTF media. A skilled artisan will appreciate that culture conditions can and will vary depending on the species of embryo. Routine optimization may be used, in all cases, to determine the best culture conditions for a particular species of embryo. In some cases, a cell line may be derived from an in vitro-cultured embryo (e.g., an embryonic stem cell line).

Alternatively, an embryo may be cultured in vivo by transferring the embryo into the uterus of a female host. Generally speaking the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and may result in a live birth of an animal derived from the embryo. Such an animal would comprise the edited chromosomal sequence encoding the inflammation-related protein in every cell of the body.

Similarly, cells comprising the introduced nucleic acids may be cultured using standard procedures to allow expression of the zinc finger nuclease. Standard cell culture techniques are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

Upon expression of the zinc finger nuclease, the chromosomal sequence may be edited. In cases in which the embryo or cell comprises an expressed zinc finger nuclease but no donor (or exchange) polynucleotide, the zinc finger nuclease recognizes, binds, and cleaves the target sequence in the chromosomal sequence of interest. The double-stranded break introduced by the zinc finger nuclease is repaired by an error-prone non-homologous end-joining DNA repair process. Consequently, a deletion, insertion or nonsense mutation may be introduced in the chromosomal sequence such that the sequence is inactivated.

In cases in which the embryo or cell comprises an expressed zinc finger nuclease as well as a donor (or exchange) polynucleotide, the zinc finger nuclease recognizes, binds, and cleaves the target sequence in the chromosome. The double-stranded break introduced by the zinc finger nuclease is repaired, via homologous recombination with the donor (or exchange) polynucleotide, such that the sequence in the donor polynucleotide is integrated into the chromosomal sequence (or a portion of the chromosomal sequence is converted to the sequence in the exchange polynucleotide). As a consequence, a sequence may be integrated into the chromosomal sequence (or a portion of the chromosomal sequence may be modified).

The genetically modified animals disclosed herein may be crossbred to create animals comprising more than one edited chromosomal sequence or to create animals that are homozygous for one or more edited chromosomal sequences. For example, two animals comprising the same edited chromosomal sequence may be crossbred to create an animal homozygous for the edited chromosomal sequence. Alternatively, animals with different edited chromosomal sequences may be crossbred to create an animal comprising both edited chromosomal sequences.

For example, animal A comprising an inactivated PPARA chromosomal sequence may be crossed with animal B comprising a chromosomally integrated sequence encoding a human PPARA to give rise to a “humanized” PPARA offspring comprising both the inactivated PPARA chromosomal sequence and the chromosomally integrated human PPARA gene. Similarly, an animal comprising an inactivated IL-4 chromosomal sequence may be crossed with an animal comprising chromosomally integrated sequence encoding the human IL-4 protein to generate “humanized” IL-4 offspring. Moreover, a humanized PPARA animal may be crossed with a humanized IL-4 animal to create a humanized PPARA/IL-4 animal. Those of skill in the art will appreciate that many combinations are possible.

In other embodiments, an animal comprising an edited chromosomal sequence disclosed herein may be crossbred to combine the edited chromosomal sequence with other genetic backgrounds. By way of non-limiting example, other genetic backgrounds may include wild type genetic backgrounds, genetic backgrounds with deletion mutations, genetic backgrounds with another targeted integration, and genetic backgrounds with non-targeted integrations.

### (IV) Applications

A further aspect of the present disclosure encompasses a method for using the genetically modified animals. In one embodiment, the genetically modified animals may be used to study the effects of mutations on the progression of inflammation using measures commonly used in the study of inflammation. Alternatively, the animals of the invention may be used to study the effects of the mutations on the progression of a disease state or disorder associated with inflammation-related proteins using measures commonly used in the study of said disease state or disorder. Non-limiting examples of measures that may be used include spontaneous behaviors of the genetically modified animal, performance during behavioral testing, physiological anomalies, differential responses to a compound, abnormalities in tissues or cells, and biochemical or molecular differences between genetically modified animals and wild type animals.

In another embodiment, the genetically modified animals and cells may be used for assessing the effect(s) of an agent on inflammation. Alternatively, the animals and cells of the invention may be used for assessing the effect(s) of an agent on the progression of a disease state or disorder associated with inflammation-related proteins. Suitable agents include without limit pharmaceutically active ingredients, drugs, food additives, pesticides, herbicides, toxins, industrial chemicals, household chemicals and other environmental chemicals, viral vectors encoding therapeutic properties, stem cell-based therapeutic agents. For example, the effect(s) of an agent may be measured in a “humanized” genetically modified rat, such that the information gained therefrom may be used to predict the effect of the agent in a human. In general, the method comprises contacting a genetically modified animal comprising at least one edited chromosomal sequence encoding an inflammation-related protein, and comparing results of a selected parameter to results obtained from contacting a control genetically modified animal with the same agent. Non limiting examples of disease states or disorders that may be associated with inflammation-related proteins include allergies, autoimmunity, arthritis, asthma, atherosclerosis, amyloid diseases, acne, cancer, infections, ischaemic heart disease, inflammatory bowel disorders, interstitial cystitis, hypersensitivities, inflammatory bowel diseases, reperfusion injury, transplant rejection, obesity, myopathies, leukopenia, vitamin deficiencies, pelvic inflammatory disease, glomeronephritis, graft versus host disease (transplant rejection), preterm labor, vasculitis, vitiligo, HIV infection and progression to AIDS.

Also provided are methods to assess the effect(s) of an agent in an isolated cell comprising at least one edited chromosomal sequence encoding an inflammation-related protein, as well as methods of using lysates of such cells (or cells derived from a genetically modified animal disclosed herein) to assess the effect(s) of an agent. For example, the role of a particular inflammation-related protein in the metabolism of a particular agent may be determined using such methods. Similarly, substrate specificity and pharmacokinetic parameter may be readily determined using such methods.

Yet another aspect encompasses a method for assessing the therapeutic efficacy of a potential gene therapy strategy. That is, a chromosomal sequence encoding an inflammation-related protein may be modified such that the inflammation is reduced or eliminated. In particular, the method comprises editing a chromosomal sequence encoding an inflammation-related protein such that an altered protein product is produced. The genetically modified animal may further be exposed to a test conditions such as exposure to a test compound, and cellular, and/or molecular responses measured and compared to those of a wild-type animal exposed to the same test conditions. Consequently, the therapeutic potential of the inflammation-related gene therapy regime may be assessed.

Still yet another aspect encompasses a method of generating a cell line or cell lysate using a genetically modified animal comprising an edited chromosomal sequence encoding an inflammation-related protein. An additional other aspect encompasses a method of producing purified biological components using a genetically modified cell or animal comprising an edited chromosomal sequence encoding an inflammation-related protein. Non-limiting examples of biological components include antibodies, cytokines, signal proteins, enzymes, receptor agonists and receptor antagonists.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

A “gene,” as used herein, refers to a DNA region (including exons and introns) encoding a gene product, as well as all DNA regions, which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The terms “polypeptide” and “protein” are used interchangeably to refer to a polymer of amino acid residues.

The term “recombination” refers to a process of exchange of genetic information between two polynucleotides. For the purposes of this disclosure, “homologous recombination” refers to the specialized form of such exchange that takes place, for example, during repair of double-strand breaks in cells. This process requires sequence similarity between the two polynucleotides, uses a “donor” or exchange molecule to template repair of a “target” molecule (i.e., the one that experienced the double-strand break), and is variously known as “non-crossover gene conversion” or “short tract gene conversion,” because it leads to the transfer of genetic information from the donor to the target. Without being bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized homologous recombination often results in an alteration of the sequence of the target molecule such that part or all of the sequence of the donor polynucleotide is incorporated into the target polynucleotide.

As used herein, the terms “target site” or “target sequence” refer to a nucleic acid sequence that defines a portion of a chromosomal sequence to be edited and to which a zinc finger nuclease is engineered to recognize and bind, provided sufficient conditions for binding exist.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations-FSwiss protein+Spupdate+PIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between regions that share a degree of sequence identity, followed by digestion with single-stranded-specific nuclease(s), and size determination of the digested fragments. Two nucleic acid, or two polypeptide sequences are substantially similar to each other when the sequences exhibit at least about 70%-75%, preferably 80%-82%, more-preferably 85%-90%, even more preferably 92%, still more preferably 95%, and most preferably 98% sequence identity over a defined length of the molecules, as determined using the methods above. As used herein, substantially similar also refers to sequences showing complete identity to a specified DNA or polypeptide sequence. DNA sequences that are substantially similar can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press). Conditions for hybridization are well-known to those of skill in the art.

Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations. With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. A particular set of hybridization conditions may be selected following standard methods in the art (see, for example, Sambrook, et al., Molecular Cloning: A Laboratory Manual, Second Edition, (1989) Cold Spring Harbor, N.Y.).

### EXAMPLES

The following examples are included to illustrate the invention.

### Example 1

### Genome Editing of CCR2 in a Model Organism

Zinc finger nuclease (ZFN)-mediated genome editing may be used to study the effects of a “knockout” mutation in an inflammation-related chromosomal sequence, such as a chromosomal sequence encoding the CCR2 protein, in a genetically modified model animal and cells derived from the animal. Such a model animal may be a rat. In general, ZFNs that bind to the rat chromosomal sequence encoding the inflammation-related protein CCR2 may be used to introduce a non-sense mutation into the coding region of the CCR2 gene, such that an active CCR2 protein may not be produced.

Capped, polyadenylated mRNA encoding the ZFN may be produced using known molecular biology techniques, including but not limited to a technique substantially similar to the technique described in Science (2009) 325:433, which is incorporated by reference herein in its entirety. The mRNA may be transfected into rat embryos. The rat embryos may be at the single cell stage when microinjected. Control embryos may be injected with 0.1 mM EDTA. The frequency of ZFN-induced double strand chromosomal breaks may be determined using the Cel-1 nuclease assay. This assay detects alleles of the target locus that deviate from wild type (WT) as a result of non-homologous end joining (NHEJ)-mediated imperfect repair of ZFN-induced DNA double strand breaks. PCR amplification of the targeted region from a pool of ZFN-treated cells may generate a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. The relative intensity of the cleavage products compared with the parental band is a measure of the level of Cel-1 cleavage of the heteroduplex. This, in turn, reflects the frequency of ZFN-mediated cleavage of the endogenous target locus that has subsequently undergone imperfect repair by NHEJ.

The development of the embryos following microinjection, and the development of inflammation-related symptoms and disorders caused by the CCR2 “knockout” may be assessed in the genetically modified rat. For CCR2, inflammation-related symptoms and disorders may include development of rheumatoid arthritis and an altered inflammatory response against tumors. The results may be compared to the control rat injected with 0.1 mM EDTA, where the chromosomal region encoding the CCR2 protein is not altered. In addition, molecular analysis of inflammation-related pathways may be performed in cells derived from the genetically modified animal comprising a CCR2 “knockout”.

### Example 2

### Generation of a Humanized Rat Expressing a Mutant Form of Human Perforin-1

Missense mutations in perforin-1, a critical effector of lymphocyte cytotoxicity, lead to a spectrum of diseases, from familial hemophagocytic lymphohistiocytosis to an increased risk of tumorigenesis. One such mutation is the V50M missense mutation where the valine amino acid at position 50 in perforin-1 is replaced with methionine. ZFN-mediated genome editing may be used to generate a humanized rat wherein the rat PRF1 gene is replaced with a mutant form of the human PRF1 gene comprising the V50M mutation. Such a humanized rat may be used to study the development of the diseases associated with the mutant human perforin-1 protein. In addition, the humanized rat may be used to assess the efficacy of potential therapeutic agents targeted at the inflammatory pathway comprising perforin-1.

The genetically modified rat may be generated using the methods described in Example 1 above. However, to generate the humanized rat, the ZFN mRNA may be co-injected with the human chromosomal sequence encoding the mutant perforin-1 protein into the rat embryo. The rat chromosomal sequence may then be replaced by the mutant human sequence by homologous recombination, and a humanized rat expressing a mutant form of the perforin-1 protein may be produced.

### Example 3

### Editing the Pten Locus

ZFNs that target and cleave the Pten locus in rats were designed and tested for activity essentially as described above in Example 1. An active pair of ZFNs was identified. The DNA binding sites were 5′-CCCCAGTTTGTGGTCtgcca-3′ (SEQ ID NO:1) and 5′-gcTAAAGGTGAAGATCTA-3′ (SEQ ID NO:2). Capped, polyadenylated mRNA encoding the active pair may be microinjected into rat embryos and the resultant embryos may be analyzed as described in Example 1. Accordingly, the Pten locus may be edited to contain a deletion or an insertion such that the coding region is disrupted and no functional gene product is made.

### Example 4

### Identification of ZFNs that Edit the Rag1 Locus

The Rag1 gene was chosen for zinc finger nuclease (ZFN) mediated genome editing. ZFNs were designed, assembled, and validated using strategies and procedures described in the examples above. ZFN design made use of an archive of pre-validated 1-finger and 2-finger modules. The rat Rag1 gene region (XM_{—}001079242) was scanned for putative zinc finger binding sites to which existing modules could be fused to generate a pair of 4-, 5-, or 6-finger proteins that would bind a 12-18 bp sequence on one strand and a 12-18 bp sequence on the other strand, with about 5-6 bp between the two binding sites. Capped, polyadenylated mRNA encoding each pair of ZFNs was produced and transfected into rat cells. Control cells were injected with mRNA encoding GFP. Active ZFN pairs were identified by detecting ZFN-induced double strand chromosomal breaks using the Cel-1 nuclease assay. This assay revealed that the ZFN pair targeted to bind 5′-ttCCTTGGGCAGTAGACctgactgtgag-3′ (SEQ ID NO:3; contact sites in upper case) and 5′-gtGACCGTGGAGTGGCAcccccacacac-3′ (SEQ ID NO: 4) cleaved within the Rag1 gene.

### Example 5

### Editing the Rag1 Locus

Capped, polyadenylated mRNA encoding the active pair of ZFNs was microinjected into fertilized rat embryos as described in the examples above. The injected embryos were either incubated in vitro, or transferred to pseudopregnant female rats to be carried to parturition. The resulting embryos/fetus, or the toe/tail clip of live born animals were harvested for DNA extraction and analysis. DNA was isolated using standard procedures. The targeted region of the Rag1 locus was PCR amplified using appropriate primers. The amplified DNA was subcloned into a suitable vector and sequenced using standard methods. FIG. 1 presents DNA sequences of edited Rag1 loci in two animals (SEQ ID NOS: 5 and 6). One animal had a 808 bp deletion in exon 2, and a second animal had a 29 bp deletion in the target sequence of exon 2. These deletions disrupt the reading frame of the Rag1 coding region.

### Example 6

### Identification of ZFNs that Edit the Rag2 Locus

ZFNs that target and cleave the Rag2 gene were identified essentially as described above. The rat Rag2 gene (XM_{—}001079235) was scanned for putative zinc finger binding sites. ZFNs were assembled and tested essentially as described in Example 1. This assay revealed that the ZFN pair targeted to bind 5′-acGTGGTATATaGCCGAGgaaaaagtgt-3′ (SEQ ID NO: 7; contact sites in uppercase) and 5′-atACCACGTCAATGGAAtggccatatct-′3′ (SEQ ID NO: 8) cleaved within the Rag2 locus.

### Example 7

### Editing the Rag2 Locus

Rat embryos were microinjected with mRNA encoding the active pair of Rag2 ZFNs essentially as described in Example 2. The injected embryos were incubated and DNA was extracted from the resultant animals. The targeted region of the Rag2 locus was PCR amplified using appropriate primers. The amplified DNA was subcloned into a suitable vector and sequenced using standard methods. FIG. 2 presents DNA sequences of edited Rag2 loci in two animals. One animal had a 13 bp deletion in the target sequence in exon 3, and a second animal had a 2 bp deletion in the target sequence of exon 3. These deletions disrupt the reading frame of the Rag2 coding region.

### Example 8

### Identification of ZFNs that Edit the FoxN 1 Locus

ZFNs that target and cleave the FoxN1 gene were identified essentially as described above in Example 1. The rat FoxN1 gene (XM_{—}220632) was scanned for putative zinc finger binding sites. ZFNs were assembled and tested essentially as described in Example 1. This assay revealed two pairs of active ZFNs that cleaved within the FoxN1 locus: a first pair targeted to bind 5′-ttAAGGGCCATGAAGATgaggatgctac-3′ (SEQ ID NO: 9; contact sites in uppercase) and 5′-caGCAAGACCGGAAGCCttccagtcagt-′3′ (SEQ ID NO: 10); and a second pair targeted to bind 5′-ttGTCGATTTTGGAAGGattgagggccc-3′ (SEQ ID NO: 11) and 5′-atGCAGGAAGAGCTGCAgaagtggaaga-′3′ (SEQ ID NO: 12)

### Example 9

### Identification of ZFNs that Edit the DNAPK Locus

ZFNs that target and cleave the DNAPK gene were identified essentially as described above in Example 1. The rat DNAPK gene (NM_{—}001108327) was scanned for putative zinc finger binding sites. ZFNs were assembled and tested essentially as described in Example 1. This assay revealed that the ZFN pair targeted to bind 5′-taCACAAGTCCtTCTCCAggagctagaa-3′ (SEQ ID NO: 13; contact sites in uppercase) and 5′-acAAAGCTTATGAAGGTcttagtgaaaa-′3′ (SEQ ID NO: 14) cleaved within the DNAPK locus.

The table below presents the amino acid sequences of helices of the active ZFNs.

| | | SEQ ID |
|---|---|---|
| Name | Sequence of Zinc Finger Helices | NO: |
| RAG1 | DRSNLSR QSGSLTR ERGTLAR RSDHLTT HKTSLKD | 15 |
| RAG1 | QNATRIK RSDALSR QSGHLSR RSADLTE DRANLSR | 16 |
| RAG2 | RSDNLSR DSSTRKK NSGNLDK QSGALAR RSDALAR | 17 |
| RAG2 | QSGNLAR RSDSLSV QSADRTK RSDTLST DRKTRIN | 18 |
| FOXN1 | TSGNLTR QSGNLAR LKQNLDA DRSHLTR RLDNRTA | 19 |
| FOXN1 | DRSDLSR QSGNLAR RSDTLSE QRQHRTT QNATRIK | 20 |
| FOXN1 | RSDHLSA QSGHLSR DSESLNA TSSNLSR DRSSRKR | 21 |
| FOXN1 | QSGSLTR QSSDLRR QRTHLTQ QSGHLQR QSGDLTR | 22 |
| DNAPK | QSGDLTR SSSDRKK DSSDRKK RSDNLST DNSNRIN | 23 |
| DNAPK | TSGHLSR QSGNLAR HLGNLKT QSSDLSR QSGNRTT | 24 |

What is claimed is:

1. A genetically modified animal comprising at least one edited chromosomal sequence encoding an inflammation-related protein.

2. The genetically modified animal of claim 1, wherein the edited chromosomal sequence is inactivated, modified, or comprises an integrated sequence.

3. The genetically modified animal of claim 1, wherein the edited chromosomal sequence is inactivated such that a functional inflammation-related protein is not produced.

4. The genetically modified animal of claim 3, wherein the inactivated chromosomal sequence comprises no exogenously introduced sequence.

5. The genetically modified animal of claim 1, wherein the edited chromosomal sequence is modified such that the inflammation-related protein is over-produced.

6. The genetically modified animal of claim 3, further comprising at least one chromosomally integrated sequence encoding a functional inflammation-related protein.

7. The genetically modified animal of claim 1, wherein the inflammation-related protein is chosen from the proteins listed in Table A, and combinations thereof.

8. The genetically modified animal of claim 1, wherein the inflammation-related protein is chosen from MCP1, CCR5, FCGR2B, FCER1g, IFN-γ, IL-4, perforin-1, COX1, COX2, TBX21, SH2BPSM1, FGFR2, SLC22A1, PPAR-α, PTEN, IL-1α, IL-1β, IL-6, IL-10, IL-12α, IL-12β, IL-13, IL-17A, IL-17B, IL-17C, IL-17D, IL-17F, IL-23, CX3CR1, CX3CL1, RAG1, PTPN22, TNFα, NOD2, CTLA4, and combinations thereof.

9. The genetically modified animal of claim 1, further comprising a conditional knock-out system for conditional expression of the inflammation-related protein.

10. The genetically modified animal of claim 1, wherein the edited chromosomal sequence comprises an integrated reporter sequence.

11. The genetically modified animal of claim 1, wherein the animal is heterozygous or homozygous for the at least one edited chromosomal sequence.

12. The genetically modified animal of claim 1, wherein the animal is an embryo, a juvenile, or an adult.

13. The genetically modified animal of claim 1, wherein the animal is chosen from bovine, canine, equine, feline, ovine, porcine, non-human primate, and rodent.

14. The genetically modified animal of claim 6, wherein the animal is rat and the chromosomally integrated sequence encoding an inflammation-related protein is human.

15. A non-human embryo, the embryo comprising at least one RNA molecule encoding a zinc finger nuclease that recognizes a chromosomal sequence encoding an inflammation-related protein, and, optionally, at least one donor polynucleotide comprising a sequence encoding an inflammation-related protein.

16. The non-human embryo of claim 15, wherein the inflammation-related protein is chosen from MCP1, CCR5, FCGR2B, FCER1g, IFN-γ, IL-4, perforin-1, COX1, COX2, TBX21, SH2BPSM1, FGFR2, SLC22A1, PPAR-α, PTEN, IL-1α, IL-1β, IL-6, IL-10, IL-12α, IL-12β, IL-13, IL-17A, IL-17B, IL-17C, IL-17D, IL-17F, IL-23, CX3CR1, CX3CL1, RAG1, PTPN22, TNFα, NOD2, CTLA4, and combinations thereof.

17. The non-human embryo of claim 15, wherein the embryo is chosen from bovine, canine, equine, feline, ovine, porcine, non-human primate, and rodent.

18. The non-human embryo of claim 15, wherein the embryo is rat and the donor polynucleotide comprising a sequence encoding an inflammation-related protein is human.

19. A genetically modified cell, the cell comprising at least one edited chromosomal sequence encoding an inflammation-related protein.

20. The genetically modified cell of claim 19, wherein the edited chromosomal sequence is inactivated, modified, or comprises an integrated sequence.

21. The genetically modified cell of claim 19, wherein the edited chromosomal sequence is inactivated such that a functional inflammation-related protein is not produced.

22. The genetically modified cell of claim 19, wherein the edited chromosomal sequence is modified such that the inflammation-related protein is over-produced.

23. The genetically modified cell of claim 21, further comprising at least one chromosomally integrated sequence encoding a functional inflammation-related protein.

24. The genetically modified cell of claim 19, wherein the inflammation-related protein is chosen from the proteins listed in Table A, and combinations thereof.

25. The genetically modified cell of claim 19, wherein the inflammation-related protein is chosen from MCP1, CCR5, FCGR2B, FCER1g, IFN-γ, IL-4, perforin-1, COX1, COX2, TBX21, SH2BPSM1, FGFR2, SLC22A1, PPAR-α, PTEN, IL-1α, IL-1β, IL-6, IL-10, IL-12α, IL-12β, IL-13, IL-17A, IL-17B, IL-17C, IL-17D, IL-17F, IL-23, CX3CR1, CX3CL1, RAG1, PTPN22, TNFα, NOD2, CTLA4, and combinations thereof.

26. The genetically modified cell of claim 19, further comprising a conditional knock-out system for conditional expression of the inflammation-related protein.

27. The genetically modified cell of claim 19, wherein the edited chromosomal sequence comprises an integrated reporter sequence.

28. The genetically modified cell of claim 19, wherein the cell is heterozygous or homozygous for the at least one edited chromosomal sequence.

29. The genetically modified cell of claim 19, wherein the cell is of bovine, canine, equine, feline, human, ovine, porcine, non-human primate, or rodent origin.

30. The genetically modified cell of claim 23, wherein the cell is of rat origin and the chromosomally integrated sequence encoding an inflammation-related protein is human.

31. A method for assessing the effect of a genetically modified inflammation-related protein on the progression or symptoms of an inflammation-related disease state in an animal, the method comprising comparing a wild type animal to a genetically modified animal comprising at least one edited chromosomal sequence encoding an inflammation-related protein, and measuring a phenotype associated with the disease state.

32. The method of claim 31, wherein the at least one edited chromosomal sequence is inactivated such that a functional inflammation-related protein is not produced.

33. The method of claim 31, wherein the at least one edited chromosomal sequence is inactivated such that the inflammation-related protein is over-produced.

34. The method of claim 31, wherein the at least one edited chromosomal sequence is inactivated such that the inflammation-related protein is not produced or is not functional, and wherein the animal further comprises at least one chromosomally integrated sequence encoding a functional inflammation-related protein.

35. The method of claim 31, wherein the inflammation-related protein is chosen from the proteins listed in Table A, and combinations thereof.

36. The method of claim 31, wherein the inflammation-related protein is chosen from MCP1, CCR5, FCGR2B, FCER1g, IFN-γ, IL-4, perforin-1, COX1, COX2, TBX21, SH2BPSM1, FGFR2, SLC22A1, PPAR-α, PTEN, IL-1α, IL-1β, IL-6, IL-10, IL-12α, IL-12β, IL-1β, IL-17A, IL-17B, IL-17C, IL-17D, IL-17F, IL-23, CX3CR1, CX3CL1, RAG1, PTPN22, TNFα, NOD2, CTLA4, and combinations thereof.

37. The method of claim 31, wherein the disease state is chosen from allergies, autoimmunity, arthritis, asthma, atherosclerosis, amyloid diseases, acne, cancer, infections, ischaemic heart disease, inflammatory bowel disorders, interstitial cystitis, hypersensitivities, inflammatory bowel diseases, reperfusion injury, transplant rejection, obesity, myopathies, leukopenia, vitamin deficiencies, pelvic inflammatory disease, glomeronephritis, graft versus host disease (transplant rejection), preterm labor, vasculitis, vitiligo, and HIV infection and progression to AIDS.

38. A method for assessing the effect of an agent on progression or symptoms of inflammation, the method comprising:
a) contacting a genetically modified animal comprising at least one edited chromosomal sequence encoding an inflammation-related protein with the agent;
b) measuring an inflammation-related phenotype, and
c) comparing results of the inflammation-related phenotype in (b) to results obtained from a control genetically modified animal comprising said edited chromosomal sequence encoding an inflammation-related protein not contacted with the agent.

39. The method of claim 38, wherein the agent is a pharmaceutically active ingredient, a drug, a toxin, or a chemical.

40. The method of claim 38, wherein the at least one edited chromosomal sequence is inactivated such that the inflammation-related protein is not produced or is not functional.

41. The method of claim 38, wherein the at least one edited chromosomal sequence is inactivated such that the inflammation-related protein is not produced or is not functional, and wherein the animal further comprises at least one chromosomally integrated sequence encoding a functional inflammation-related protein.

42. The method of claim 38, wherein the inflammation-related protein is chosen from the proteins listed in Table A, and combinations thereof.

43. The method of claim 38, wherein the inflammation-related protein is chosen from MCP1, CCR5, FCGR2B, FCER1g, IFN-γ, IL-4, perforin-1, COX1, COX2, TBX21, SH2BPSM1, FGFR2, SLC22A1, PPAR-α, PTEN, IL-1α, IL-1β, IL-6, IL-10, IL-12α, IL-12β, IL-13, IL-17A, IL-17B, IL-17C, IL-17D, IL-17F, IL-23, CX3CR1, CX3CL1, RAG1, PTPN22, TNFα, NOD2, CTLA4, and combinations thereof.
